(19) United States
(12) Patent Application Publication
(10) Pub. No.: US 2022/0401575 A1
(43) Pub. Date: Dec. 22, 2022

(54) COMPOSITIONALLY DEFINED PLASMID DNA/POLYCATION NANOPARTICLES AND METHODS FOR MAKING THE SAME

(71) Applicant: The Johns Hopkins University, Baltimore, MD (US)

(72) Inventors: Hai-Quan Mao, Baltimore, MD (US); Yizong Hu, Baltimore, MD (US); Martin Gilbert Pomper, Baltimore, MD (US); Heng-wen Liu, Baltimore, MD (US); Il Minn, Baltimore, MD (US); Christopher Ullman, Baltimore, MD (US); Christine Carrington, Baltimore, MD (US)

(21) Appl. No.: 17/606,605

(22) PCT Filed: Apr. 29, 2020
(86) PCT No.: PCT/US2020/030429

§ 371 Date: Oct. 26, 2021

Related U.S. Application Data
(60) Provisional application No. 62/840,152, filed on Apr. 29, 2019.

(51) Int. Cl. A61K 47/69; A61K 48/00; A61K 9/51; C12N 15/113; C12N 15/88

(52) U.S. Cl. CPC A61K 47/6929; A61K 48/0091; A61K 9/5146; C12N 15/113; C12N 15/88

(57) ABSTRACT

The presently disclosed subject matter provides a kinetically controlled mixing process, referred to herein as “flash nanocomplexation” or “(FNC),” to accelerate the mixing of a polyanion solution, for example, a plasmid DNA solution, with a polycation solution to match the polyelectrolyte complex (PEC) assembly kinetics through turbulent mixing in a microchamber, thus achieving explicit control of the kinetic conditions for nanoparticle assembly as demonstrated by the tunability of nanoparticle size, composition, hydrodynamic size, hydrodynamic density, surface charge, and polyanion payload.

### FEDERALLY SPONSORED RESEARCH OR DEVELOPMENT

This invention was made with government support under EB018358 and EB024495 awarded by the National Institutes of Health. The government has certain rights in the invention.

### BACKGROUND

Polyelectrolyte complex (PEC) formation has been widely used to assemble particulate vehicles for delivery of a wide range of macromolecular therapeutics including plasmid DNA (pDNA), messenger RNA (mRNA), small interfering RNA (siRNA), proteins, and peptides. As a promising non-viral gene delivery approach, pDNA molecules are condensed and packaged into PEC nanoparticles using a polycationic carrier in an aqueous solution. The assembled pDNA/polycation nanoparticles facilitate transport and access to target cells and cellular compartments, and protect pDNA from enzymatic degradation. Shi et al., 2017. The in vivo fate and efficacy for gene delivery and rate of transfection of the nanoparticles, as revealed by many recent efforts, are dependent on nanoparticle characteristics, such as their size range and distribution, Hickey et al., 2015, morphology, Williford et al., surface properties, compositions, and structures. Blanco et al., 2015. It has been challenging, however, to uncover detailed understandings of the relationship between these nanoparticle characteristics and their interactions with biological systems. This challenge is largely due to the lack of sufficient control over the PEC assembly kinetics, which dictates the characteristics of the assembled nanoparticles.

### SUMMARY

In some aspects, the presently disclosed subject matter provides a method for preparing uniform polyelectrolyte complex (PEC) nanoparticles, the method comprising homogeneously mixing one or more water-soluble polycationic polymers with one or more water-soluble polyanionic polymers under conditions having a characteristic assembly time (τ_{A}), over which assembly of the PEC nanoparticles occurs, greater than a characteristic mixing time (τ_{M}), over which the one or more water-soluble polycationic polymers and the one or more water-soluble polyanionic polymers are mixed homogenously.

In particular aspects, the method comprises a flash nanocomplexation (FNC) method. In such aspects, the method comprises continuously generating uniform polyelectrolyte complex (PEC) nanoparticles by: (a) flowing a first stream comprising one or more water-soluble polycationic polymers at a first variable flow rate into a confined chamber; (b) flowing a second stream comprising one or more water-soluble polyanionic polymers at a second variable flow rate into the confined chamber, wherein the first stream and the second stream are on opposing sides when entering the confined chamber; and (c) optionally flowing a third stream comprising one or more components selected from the group consisting of one or more water-soluble therapeutic agents, one or more miscible organic solvents, and/or one or more cryoprotectants at a third variable flow rate into the confined chamber; wherein each stream is equidistant from the other two streams when entering the confined chamber; wherein the first variable flow rate, the second variable flow rate, and the third variable flow rate, if present, can be the same or different; and (d) impinging the first stream, the second stream, and the third stream, if present, in the confined chamber until the Reynolds number is from about 1,000 to about 20,000, thereby causing the one or more water-soluble polycationic polymers and the one or more water-soluble polyanionic polymers to undergo a polyelectrolyte complexation process that continuously generates PEC nanoparticles, wherein the polyelectrolyte complexation process occurs under conditions having a characteristic assembly time (τ_{A}), over which assembly of the PEC nanoparticles occurs, which is greater than a characteristic mixing time (τ_{M}), over which components of the first stream, second stream, and third stream, if present, are mixed homogenously.

In certain aspects, the first variable flow rate, the second variable flow rate, and the third variable flow rate, if present, are each equal to or greater than about 3 milliliters/minute (mL/min). In particular aspects, the first variable flow rate, the second variable flow rate, and the third variable flow rate, if present, are each between about 3 mL/min to about 50 mL/min.

In certain aspects, the characteristic mixing time is between about 1 ms to about 200 ms. In particular aspects, the characteristic mixing time is about 15 ms.

In some aspects, the Reynolds number has a range from about 2,000 to about 8,000 or from about 3,000 to about 5,000. In some aspects, the pH value of the first stream and the pH value of the second stream each has a range from about 2.5 to about 8.4. In particular aspects, the pH value of the first stream and the pH value of the second stream each is about 3.5.

In some aspects, the one or more water-soluble polycationic polymers are selected from the group consisting of chitosan, PAMAM dendrimers, polyethylenimine (PEI), protamine, poly(arginine), poly(lysine), poly(beta-aminoesters), cationic peptides and derivatives thereof.

In some aspects, the one or more water-soluble polyanionic polymers are selected from the group consisting of poly(aspartic acid), poly(glutamic acid), negatively charged block copolymers, heparin sulfate, dextran sulfate, hyaluronic acid, alginate, tripolyphosphate (TPP), oligo(glutamic acid), a cytokine, a protein, a peptide, a growth factor, and a nucleic acid. In particular aspects, the nucleic acid is selected from the group consisting of an antisense oligonucleotide, cDNA, genomic DNA, guide RNA, plasmid DNA, vector DNA, mRNA, miRNA, piRNA, shRNA, and siRNA.

In further aspects, the first stream and/or the second stream further comprise one or more water-soluble therapeutic agents. In certain aspects, the one or more water-soluble therapeutic agents are selected from the group consisting of a small molecule, carbohydrate, sugar, protein, peptide, nucleic acid, antibody or antibody fragment thereof, hormone, hormone receptor, receptor ligand, cytokine, and growth factor.

In certain aspects, the one or more water-soluble polyanionic polymers is plasmid DNA and the one or more water-soluble polycationic polymers is linear polyethylenimine (PEI) or a derivative thereof. In such aspects, the plasmid DNA concentration is between about 25 to about 800 μg/mL. In particular aspects, the plasmid concentration is selected from the group consisting of about 25 μg/mL, about 50 μg/mL, about 100 μg/mL, about 200 μg/mL, about 400 μg/mL, and about 800 μg/mL.

In other aspects, the presently disclosed subject matter provides a uniform polyelectrolyte complex (PEC) nanoparticle or plurality of PEC nanoparticles generated from the presently disclosed method.

In some aspects, the PEC nanoparticle has an average of about 1 to about 50 copies of pDNA per nanoparticle. In particular aspects, the PEC nanoparticle has an average of about 1.7 to about 21.8 copies of pDNA per nanoparticle; about 1.7 to about 3.5 copies of pDNA per nanoparticle; about 1.7 to about 5.0 copies of pDNA per nanoparticle; about 1.7 to about 6.1 copies of pDNA per nanoparticle; about 1.7 to about 8.0 copies of pDNA per nanoparticle; about 1.7 to about 8.5 copies of pDNA per nanoparticle; about 1.7 to about 9.1 copies of pDNA per nanoparticle; about 1.7 to about 9.5 copies of pDNA per nanoparticle; about 1.7 copies of pDNA per nanoparticle; about 3.5 copies of pDNA per nanoparticle; about 4.4 copies of pDNA per nanoparticle; about 5.0 copies of pDNA per nanoparticle; about 6.1 copies of pDNA per nanoparticle; about 8.0 copies of pDNA per nanoparticle; about 8.1 copies of pDNA per nanoparticle; about 8.5 copies of pDNA per nanoparticle; about 9.1 copies of pDNA per nanoparticle; about 9.5 copies of pDNA per nanoparticle; or about 21.8 pDNA copies per nanoparticle. In yet more particular aspects, the PEC nanoparticle has one pDNA per nanoparticle.

In some aspects, the PEC nanoparticle has an average size between about 35 nm to about 130 nm. In particular aspects, the PEC nanoparticle has an average size of about 80 nm.

In certain aspects, the PEC nanoparticle comprises polyethylenimine and plasmid DNA. In such aspects, the PEC nanoparticle has a ratio of amine in the polyethylenimine to phosphate in the plasmid DNA (N/P) between about 3 to about 10. In particular aspects, the PEC nanoparticle has an NIP selected from the group consisting of about 3, about 4, about 5, about 6, about 7, about 8, about 9, and about 10.

In some aspects, the PEC nanoparticle has a percentage of bound lPEI to total amount of lPEI between about 50% to about 75%. In certain aspects, the plurality of PEC nanoparticles has a polydispersity index (PDI) between about 0.1 and about 0.25. In certain aspects, the PEC nanoparticle has a surface charge between about +20 to about +50 mV. In certain aspects, the PEC nanoparticle has an apparent hydrodynamic density between about 60 Da/nm³ to about 80 Da/nm³, depending on the medium used to suspend the nanoparticles.

In other aspects, the presently disclosed subject matter provides a formulation comprising the presently disclosed PEC nanoparticle or plurality of PEC nanoparticles. In particular aspects, the formulation comprises a lyophilized formulation. In certain aspects, the PEC nanoparticle or plurality of PEC nanoparticles exhibits long term stability at −20° C. for at least 9 months.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below

### BRIEF DESCRIPTION OF THE DRAWINGS

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1A and FIG. 1B depict diagrams of representative embodiments of the presently disclosed confined impinging jet (CIJ) device. FIG. 1A depicts an embodiment of the CIJ device used to fabricate polyelectrolyte complex (PEC) nanoparticles under rapid mixing conditions. Streams are independently loaded with linear polyethylenimine (lPEI) and plasmid DNA, and lPEI-DNA complex nanoparticles are formed in a small confined chamber before being collected; FIG. 1B depicts a schematic diagram showing a CIJ device with 3-jets separated by a 120° angle. Jet 1 can be loaded with positively charged polymers including chitosan, PAMAM dendrimers, PEI, protamine sulfate, poly(arginine, poly(lysine) and positively charged block copolymers. Jet 2 is charged with negatively charged macromolecules including poly(aspartic acid), heparin sulfate, dextran sulfate, hyaluronic acid, tripolyphosphate, oligo(glutamic acids), cytokines, proteins, peptides, growth factors, DNA, siRNA, mRNA. Jet 3 can be either capped or loaded with water miscible organic solvents to control the polarity of the final formulation in situ (prior art; U.S. Patent Application Publication No. 20170042829, for METHODS OF PREPARING POLYELECTROLYTE COMPLEX NANOPARTICLES, to Mao et al., published Feb. 16, 2017, which is incorporated herein by reference in its entirety);

FIG. 2A, FIG. 2B, FIG. 2C, FIG. 2D, FIG. 2E, FIG. 2F, and FIG. 2G show the effect of characteristic mixing time τ_{M} on pDNA/lPEI nanoparticle assembly. (FIG. 2A, FIG. 2B) Effect of mixing kinetics profile (τ_{A} and flow rate Q) on the average nanoparticle size Dg (FIG. 2A) and uniformity shown as the size distribution width given by DLS (FIG. 2B). The mixing kinetics scale is divided into two regions: Region I (τ_{M}<τ_{A}) and Region II (τ_{M}>τ_{A}). Labels 1, 2 and 3 denote three representative preparations generated from three different mixing conditions; (FIG. 2C) Transmission electron microscopy (TEM) images and DLS profiles of the three sets of nanoparticles prepared at Q=1.25 mL/min, τ_{M}=1.8×10⁵ ms (Prep. 1), Q=5 mL/min, =790 ms (Prep. 2), and Q=20 mL/min, τ_{M}=15 ms (Prep. 3). Scale bar=50 nm (for left panel) and 200 nm (for right panel); (FIG. 2D, FIG. 2E) Effect of input pDNA concentration and plasmid size on the average nanoparticle size Dg (FIG. 2D) and zeta potential (FIG. 2E) prepared by τ_{M}=15 ms; (FIG. 2F, FIG. 2G) Effect of N/P ratio on the average nanoparticle size Dg (FIG. 2F) and zeta potential (FIG. 2G) prepared by τ_{M}=15 ms. For the conditions tested, the size profile and zeta potential of pDNA/lPEI nanoparticles did not vary with the N/P ratio from 4 to 6;

FIG. 3A, FIG. 3B, FIG. 3C, FIG. 3D, and FIG. 3E show compositions of the FNC-assembled pDNA/lPEI nanoparticles. (FIG. 3A) The fraction of bound lPEI and the composition of the assembled nanoparticles remained similar when nanoparticles were prepared at different input pDNA concentrations or with different plasmids; (FIG. 3B) Bound vs. free lPEI amount and proportions with an input N/P ratio from 3 to 6 for gWiz-Luc and gWiz-GFP nanoparticle formulations assembled under a turbulent mixing condition (Q=20 mL/min, τ_{M}=15 ms<τ_{A}) and a laminar mixing condition (Q=5 mL/min, τ_{M}=790 ms>τ_{A}). Labels: Luc and GFP for gWiz-Luc and gWiz-GFP plasmid nanoparticles, respectively; (FIG. 3C) Bound lPEI fraction and zeta-potential of nanoparticles prepared with 50 or 200 μg/mL of gWiz-Luc pDNA under different flow rates, suggesting that all gWiz-Luc/lPEI nanoparticles share the same average composition; (FIG. 3D) A representative Zimm plot for I2/lPEI nanoparticles with a molar mass of 5.32×10⁷ Da, also showing the second viral coefficient A₂ approaching zero; and (FIG. 3E) Representative Debye plots for gWiz-GFP/lPEI nanoparticles prepared by varied input concentration of I₂ plasmid.

FIG. 4A, FIG. 4B, FIG. 4C, and FIG. 4D, and FIG. 4E show assembly of pDNA/lPEI PEC nanoparticles. (FIG. 4A, FIG. 4B) Correlation of nanoparticle average molar mass and size (FIG. 4A) and radius of gyration (FIG. 4B) for nanoparticles assembled under turbulent mixing condition (Q=20 mL/min, τ_{M}=15 ms). Each data point in (FIG. 4A) and (FIG. 4B) represents an independent formulation batch; (FIG. 4C) Application of the linear fits from Eq. 2 (Upper panel) and Eq. 3 (Bottom panel) to nanoparticles formulated with different N/P ratios at Q=20 mL/min; (FIG. 4D) Correlation of nanoparticle average molar mass and size for nanoparticles produced by different mixing conditions, i.e. with different τ_{M}. For input pDNA concentration of 25 μg/mL (orange), label 1 to 8 represent τ_{M} of 7, 11, 15, 23, 163, 5855, 4×10⁵ ms, and pipetting respectively; for 100 μg/mL (blue), label 1 to 6 represent τ_{M} of 8, 15, 42, 795, 10⁴ and 2×10⁵ ms, respectively; (FIG. 4E) The proposed two-step pDNA/lPEI PEC nanoparticle assembly model under turbulent mixing condition (τ_{M}<TA);

FIG. 5A, FIG. 5B, FIG. 5C, and FIG. 5D show the transfection process and efficiency of pDNA/lPEI nanoparticles with different numbers of pDNA per particle. (FIG. 5A) Cellular uptake quantitative assay of nanoparticles prepared with 3H-labeled pDNA in PC3 prostate cell line following a 4-h incubation period (pDNA dosage=0.6 μg/10⁴ cells); (FIG. 5B) The in vitro transfection efficiency of nanoparticles with different N in PC3 cells with a 4-h incubation (pDNA dosage=0.6 μg/10⁴ cells). The asterisks denote significance level when comparing with N=6.1 nanoparticle group; (FIG. 5C) The in vivo transfection efficiency (bioluminescence radiance) in the lung of Balb/c mice at 12 h post i.v. injection of nanoparticles (dose=30 μg pDNA per mouse); (FIG. 5D) Whole-body biodistribution of nanoparticles at 1-h post i.v. injection of ³H-labeled nanoparticles containing 30 μg pDNA per mouse. Labels: H: heart, K: kidneys, S: stomach, SI: small intestine. For statistical analysis, *p<0.05, **p<0.01, and ***p<0.001 from one-way ANOVA and multiple comparisons;

FIG. 6A, FIG. 6B, FIG. 6C, FIG. 6D, and FIG. 6E show the transgene expression of pDNA/lPEI nanoparticles produced under kinetically controlled conditions with different N/P ratios and payload levels (N). (FIG. 6A) In vitro transfection efficiencies of nanoparticles (W1-8, see Table 4) in PC3 cancer cell line (dose=0.6 μg gWiz-Luc plasmid/10⁴ cells); (FIG. 6B) In vivo transfection efficiency in the lung in healthy Balb/c mice at 12 h post i.v. injection of nanoparticles (W1-8, see Table 4) containing 40 μg gWiz-Luc plasmid per mouse (left) and representative IVIS images of groups with significant differences in transgene expression (right); (FIG. 6C) In vivo transfection efficiency in the lung of an LL2 metastasis model in the NSG mice at 48 h post injection of nanoparticles (P1-8, see Table 4) containing 40 μg PEG-Luc plasmid per mouse (left) and representative IVIS images of groups with significant differences in transgene expression (right); (FIG. 6D) Whole-body biodistributions in Balb/c mice at 1 h post injection of nanoparticles (W1, W2, W6, W8) containing 40 μg 3H-labeled gWiz-Luc plasmid per mouse. Labels: H: heart, K: kidneys, S: stomach, SI: small intestine; (FIG. 6E) Biodistributions to the lung of mice shown in (FIG. 6D);

FIG. 7A, FIG. 7B, FIG. 7C, FIG. 7D, and FIG. 7E show the scale-up production of off-the-shelf pDNA/lPEI nanoparticles and the long-term storage stability. (FIG. 7A) Lyophilization and reconstitution of nanoparticles prepared using FNC setup; (FIG. 7B) Nanoparticle characteristics upon reconstitution of lyophilized nanoparticles stored at −20° C. at Months 0, 1, 3, 6 and 9. Month 0 represents a reconstituted sample right after completion of lyophilization;

FIG. 8A, FIG. 8B, FIG. 8C, and FIG. 8D show size distributions of PEC nanoparticles formulated with different input pDNA concentrations and input N/P ratios with τ_{M}<TA. (FIG. 8A) Size distributions and (FIG. 8B) polydispersity index (PDI) of nanoparticles prepared by different input pDNA concentrations; (FIG. 8A) Size distributions and (FIG. 8B) polydispersity index (PDI) of nanoparticles prepared by different input N/P ratios;

FIG. 9A and FIG. 9B and show TEM images for nanoparticles prepared by different input pDNA concentrations and N/P ratios. TEM images of (FIG. 9A) gWiz-Luc PEC nanoparticles prepared with an input pDNA concentration of 50 and 800 μg/mL. Note that the TEM images of gWiz-Luc PEC nanoparticles prepared with an input pDNA concentration of 200 μg/mL are shown in FIG. 2C. These TEM observations show the uniformity of the pDNA/in vivo-jetPEI® nanoparticles prepared under turbulent mixing conditions at impinging flow rate of Q=20 mL/min; (FIG. 9B) gWiz-GFP PEC nanoparticles prepared with input N/P ratio of 3 or 6, demonstrating similarity of size across preparations with different N/P ratios. Scale bar=50 nm (for left two penal) and 200 nm (for right panel);

FIG. 10A, FIG. 10B, FIG. 10C, FIG. 10E, and FIG. 10F show non-uniform PEC nanoparticles produced by pipetting method without tunability of size by input pDNA concentrations. The size of the PEC nanoparticles made by (FIG. 10A) I2 plasmid; (FIG. 10B) gWiz-GFP plasmid; (FIG. 10C) gWiz-Luc plasmid; and polydispersity index (PDI) of PEC nanoparticles made from (FIG. 10D) I2 plasmid; (FIG. 10E) gWiz-GFP plasmid; (FIG. 10F) gWiz-Luc plasmid. Labels: B1, B2, B3 and B4 represent 4 different procedures followed to make nanoparticles by pipetting, see Table 2;

FIG. 11 shows the determination of pDNA concentration in PEC nanoparticle suspensions. Upon PEI binding and assembly, the absorbance at 260 nm by pDNA molecules increases but still follow a linear relationship with respect to pDNA concentrations. This standard curve was used to assess pDNA concentrations in any PEC nanoparticle suspensions;

FIG. 12A, FIG. 12B, FIG. 12C, FIG. 12D, FIG. 12E, and FIG. 12F show SLS data for nanoparticles prepared by a flow rate of 20 mL/min with different input pDNA concentrations and N/P ratios. Full Zimm plots of gWiz-Luc PEC nanoparticles with (FIG. 12A) a molar mass of 1.02×10⁷ Da, and 1.7 pDNAs per nanoparticle; (FIG. 12B) a molar mass of 3.59×10⁷ Da, and 6.1 pDNAs per nanoparticle; (FIG. 12C) a molar mass of 1.27×10⁸ Da, and 21.8 pDNAs per nanoparticle, demonstrating a universal zero second virial coefficient; Combined Debye plots for PEC nanoparticles prepared with different input pDNA concentrations by (FIG. 12D) I2 plasmid and (FIG. 12E) gWiz-Luc plasmid; or (FIG. 12F) different input N/P ratios by gWiz-Luc plasmid. For (FIG. 12A), the original 34 μg/mL (total mass concentration in nanoparticle) sample was concentrated to 81 μg/mL and then diluted to have 54 μg/mL. The molar mass seemed to have decreased slightly upon concentration. But overall, the system presented a second virial coefficient that is equal to 0;

FIG. 13 is a standard curve for quantitative assessment of absolute amount of ³H-labeled pDNA in biological samples. Different amount of ³H-labeled pDNA solutions were added into 4 mL scintillation fluid contained in 7-mL glass scintillation vials. The same readings procedures were applied as described in Section 1.6 to obtain the standard curve. All readings of real biological samples (cell lysate or mouse tissue solute) fell into the quantity range shown in this standard curve;

FIG. 14A and FIG. 14B show in vivo transfection efficiency in lungs upon dosing of PEC nanoparticles with different average pDNA copy number per nanoparticle (N̅). (FIG. 14A) IVIS whole-body bioluminescence images of all groups at 12-h post injection of nanoparticles containing 30 μg pDNA per mouse. Scale bar: local radiance with unit of 10⁻⁶ photon/s/cm²/sr; (FIG. 14B) Luciferase abundance as measured in homogenized lungs in 3 mice with highest signals, showing a perceived trend that PEC nanoparticles with a higher N gave better transfection efficiency in lungs;

FIG. 15A, FIG. 15B, and FIG. 15C show biodistribution of dosed PEC nanoparticles with different pDNA copy number per nanoparticle (N̅). The abundance of delivered pDNA in (FIG. 15A) lungs; (FIG. 15B) liver; and (FIG. 15C) spleen;

FIG. 16 shows the correlation between IVIS region of interest (ROI) quantitative results and luciferase abundance in tissue. The IVIS ROI quantitative analysis was done with an exposure time of 30 seconds to the lung area with mice dosed with PEC nanoparticles with different N as shown in FIG. 15. The lungs were harvested from the mice immediately after the imaging and were homogenized in luciferase assay report lysis buffer (Promega, US) by a sonication probe to release the luciferase protein. Then the luciferase quantity in the tissue sample was determined as described in Section 1.6 for in vitro transfection efficiency assessment;

FIG. 17A, FIG. 17B, and FIG. 17C show in vivo transfection efficiencies of PEC nanoparticles with different pDNA payload and PEI compositions prepared by kinetically controlled conditions in healthy Balb/c mice. (FIG. 17A) IVIS whole-body images of all groups dosed with formulations listed in Table 4 at 12, 24 and 48-h post injection of PEC nanoparticles containing 40 μg pDNA per mouse. The label D denotes died mouse due to toxicity; Scale bar: local radiance with unit of 10⁻⁷ photon/s/cm²/sr; and the IVIS ROI quantitative analysis results at (FIG. 17B) 24-h and (FIG. 17C) 48-h post-injection;

FIG. 18A and FIG. 18B show tumor-specific transfection and expression efficiencies of PEC nanoparticles with different pDNA payload and PEI compositions prepared by kinetically controlled conditions in a LL2 lung metastasis model on NSG mice. (FIG. 18A) IVIS whole-body images of all groups dosed with formulations listed in Table 4 at 48-h and 72-h post injection of PEC nanoparticles containing 40 m pDNA per mouse. Scale bars: local radiance with unit of 10⁻⁵ photon/s/cm²/sr; (FIG. 18B) IVIS ROI quantitative analysis results at 72-h post-injection time point;

FIG. 19A and FIG. 19B show biodistribution data of PEC nanoparticle formulations with significant findings in transfection and transgene activities. (FIG. 19A) pDNA abundance in liver; (FIG. 19B) pDNA abundance in spleen;

FIG. 20A shows three independent experiments preparing nanoparticles via pipetting, exhibiting no reproducibility;

FIG. 20B shows three independent experiments preparing nanoparticles via the presently disclosed FNC-assembly method, exhibiting excellent reproducibility;

FIG. 21A shows nanoparticles prepared via pipetting, which were monitored for 1-h post preparation and which show severe aggregation; and

FIG. 21B shows FNC-assembled nanoparticles monitored for 96-h post-preparation, showing good stability.

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

The presently disclosed subject matter provides a flash nanocomplexation (FNC) method for producing polyelectrolyte complex nanoparticles in a continuous and scalable manner. The presently disclosed FNC method generates nanoparticles as a result of polyelectrolyte complexation without relying on solvent-induced supersaturation of copolymers. The polyelectrolyte complex nanoparticles produced by FNC have a smaller size, better uniformity and lower polydispersity than polyelectrolyte complexes prepared using conventional methods. For example, compared to bulk preparation methods, the FNC process allows for the formation of uniform nanoparticles with tunable size in a continuous flow operation process, which is amenable for scale-up production. FNC also offers a higher degree of versatility and control over particle size and distribution, higher drug encapsulation efficiency, and improved colloidal stability (Shen et al., 2011; D'Addio et al., 2013; D'Addio et al., 2102; Gindy et al., 2008; Lewis et al., 2015; D'Addio et al., 2011; Luo et al., 2014; Santos et al., 2014).

Furthermore, the presently disclosed methods result in condensed and compact polyelectrolyte nanoparticles through improved polymer chain entanglement. In addition, the methods provide a means to efficiently encapsulate therapeutic agents, such as proteins or nucleic acids, in polyelectrolyte nanoparticles while retaining their intrinsic physiochemical properties. Furthermore, formulations of DNA-containing nanoparticles prepared with these novel methods have improved particle size and shape distribution, and exhibit higher cell transfection efficiency when compared to bulk preparation methods.

### I. Compositionally Defined Plasmid DNA/Polycation Nanoparticles and Methods for Making the Same

In some embodiments, the presently disclosed subject matter provides a method for preparing uniform polyelectrolyte complex (PEC) nanoparticles, the method comprising homogeneously mixing one or more water-soluble polycationic polymers with one or more water-soluble polyanionic polymers under conditions having a characteristic assembly time (TA), over which assembly of the PEC nanoparticles occurs, greater than a characteristic mixing time (τ_{M}), over which the one or more water-soluble polycationic polymers and the one or more water-soluble polyanionic polymers are mixed homogenously.

In particular embodiments, the method comprises a flash nanocomplexation (FNC) method. In such aspects, the method comprises continuously generating uniform polyelectrolyte complex (PEC) nanoparticles by: (a) flowing a first stream comprising one or more water-soluble polycationic polymers at a first variable flow rate into a confined chamber; (b) flowing a second stream comprising one or more water-soluble polyanionic polymers at a second variable flow rate into the confined chamber, wherein the first stream and the second stream are on opposing sides when entering the confined chamber; and (c) optionally flowing a third stream comprising one or more components selected from the group consisting of one or more water-soluble therapeutic agents, one or more miscible organic solvents, and/or one or more cryoprotectants at a third variable flow rate into the confined chamber; wherein each stream is equidistant from the other two streams when entering the confined chamber; wherein the first variable flow rate, the second variable flow rate, and the third variable flow rate, if present, can be the same or different; and (d) impinging the first stream, the second stream, and the third stream, if present, in the confined chamber until the Reynolds number is from about 1,000 to about 20,000, thereby causing the one or more water-soluble polycationic polymers and the one or more water-soluble polyanionic polymers to undergo a polyelectrolyte complexation process that continuously generates PEC nanoparticles, wherein the polyelectrolyte complexation process occurs under conditions having a characteristic assembly time (TA), over which assembly of the PEC nanoparticles occurs, which is greater than a characteristic mixing time (τ_{M}), over which components of the first stream, second stream, and third stream, if present, are mixed homogenously.

As used herein “polyelectrolyte complexes” (also known as polyelectrolyte coacervates or “PECs”) are the association complexes formed between oppositely charged particles (e.g., polymer-polymer, polymer-drug, and polymer-drug-polymer). Polyelectrolyte complexes are formed due to electrostatic interaction between oppositely charged polyions, i.e. water-soluble polycations and water-soluble polyanions. As used herein, the term “continuously” refers to a process that is uninterrupted in time, such as the generation of PEC nanoparticles while at least two presently disclosed streams are flowing into a confined chamber. As used herein, the term “water-soluble” refers to the ability of a compound to be able to be dissolved in water.

In some embodiments, the water-soluble polyions are dissolved in a suitable solvent, resulting in elementary charges distributed along the macromolecular chains. In various embodiments, polyelectrolyte complexes are formed when macromolecules of opposite charge are allowed to interact. For example, in some embodiments, flash precipitated nanoparticles of polyelectrolyte complexes are formed by rapidly and homogenously mixing streams, i.e., a water-soluble polycation dissolved in a stream and a water-soluble polyanion dissolved in a stream.

In some embodiments, the streams are compositions that include one or more fluid components and are capable of carrying a solid or solids in solution or suspension. Typically, the streams are polar, e.g. acetic acid or water. More typically, the stream is water.

The streams are impinged in the confined chamber until the Reynolds number is from about 1,000 to about 20,000, thereby causing the water-soluble polycationic polymers and the water-soluble polyanionic polymers to undergo a polyelectrolyte complexation process that continuously generates PEC nanoparticles. As used herein, the term “impinging” refers to at least two streams striking each other in the confined chamber at a high flow rate. Using the presently disclosed methods and device, it has been surprisingly shown that a molecule, such as a DNA molecule, remains intact under such high shear conditions.

The rapid and homogenous mixing of the first and second streams to generate polyelectrolyte complex nanoparticles, for example, can be achieved through various methods during which the flow rate and the mixing efficiency and velocity is controlled. In some embodiments, polyelectrolyte complex nanoparticles may be produced by flash nanocomplexation using a centripetal mixer or a batch flash mixer. See, for example, Johnson et al., U.S. Patent Application Publication No. 2004/0091546, which is herein incorporated by reference in its entirety.

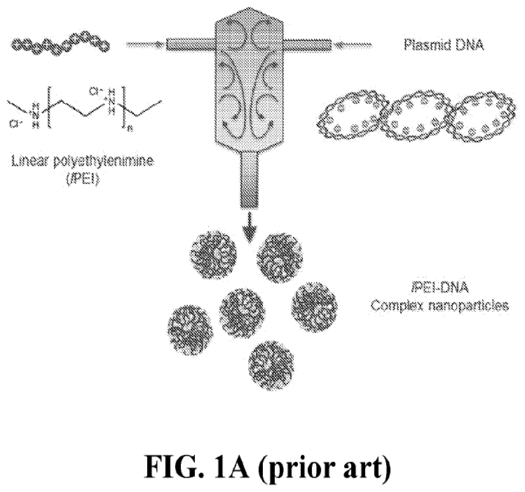

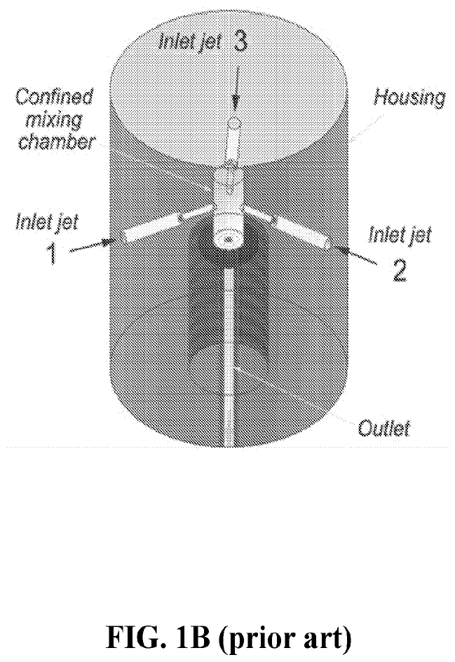

As another example, the mixing of the first and second streams may be accomplished using a confined impinging jet (CIJ) device with at least two high-velocity jets (FIG. 1A, FIG. 1B, FIG. 1C), which is disclosed in U.S. Patent Application Publication No. 20170042829, for METHODS OF PREPARING POLYELECTROLYTE COMPLEX NANOPARTICLES, to Mao et al., published Feb. 16, 2017, which is incorporated herein by reference in its entirety. In a typical embodiment, oppositely charged streams are loaded into separated syringes and fed into a confined chamber of a CIJ device by digitally controlled syringe pumps (e.g., New Era Pump System, model NE-4000). In some embodiments, a long tube runner serving as an outlet is used to ensure that the opposing streams brought into the confined chamber are fully reacted before collection. In some embodiments, the first stream and the second stream are on opposing sides when entering the confined chamber. As used herein, the term “opposing sides” means that the streams are generally opposite each other. In some embodiments, the streams are directly opposite each other. In some embodiments, the streams may not be directly opposite each other.

Methods of the present disclosure also include providing one or more additional streams. For example, in some embodiments, the method could include providing a third stream comprising a further additive such as a therapeutic agent as described herein below, a saline solution, a water miscible organic solvent (e.g., dimethyl sulfoxide, dimethyl formamide, acetonitrile, tetrahydrofuran, methanol, ethanol, isopropanol), to control the polarity of the final formulation in situ, or a cryoprotectant (e.g., glycerol, trehalose, sucrose, dextrose) to improve the colloidal stability of the nanoparticles upon reconstitution. In some embodiments, a third, fourth or even further numbers of jets may be added to a CIJ device to accommodate additional streams with additives such as those described herein.

In some embodiments, the presently disclosed methods further comprise flowing a third stream into the confined chamber, wherein each stream is equidistant from the other two streams when entering the confined chamber. In some embodiments, keeping the streams equidistant from each other allows even mixing of the streams to occur.

In some embodiments, the pH value of the first stream and the pH value of the second stream range from about 2.5 to about 8.4, including 2.5, 3.0, 3.5, 4.0, 4.5, 5.0, 5.5, 6.0, 6.5, 7.0, 7.5, 8.0, and 8.4. In some embodiments, the pH value of the first stream and the pH value of the second stream range from about 3.5 to about 7.4, including 3.5, 4.0, 4.5, 5.0, 5.5, 6.0, 6.5, 7.0, and 7.4. In some embodiments, the pH value of the first stream and the pH value of the second stream are each 3.5.

The flow rate at which the streams contained in the syringes of the above described CIJ device may be impinged into the confined chamber may be readily tuned via the programmable syringe pumps, for example. Further, in some embodiments, the characteristic mixing time is a function of the flow rate and can be adjusted by changing the flow rate. For instance, at high flow rates, the flow pattern may assume turbulent-like characteristics and the mixing time may be in the order of a few milliseconds. Under these conditions, efficient mass transfer is achieved, and discrete and uniform nanoparticles with a narrow size distribution may be produced. In various embodiments, the final average particle size is a function of the mixing time, the concentration and the chemical composition of the polyelectrolytes.

The mixing efficiency and the nature of the flow, which influence the mixing velocity, is commonly defined by the Reynold's number (Re), a dimensionless number representing the ratio of the inertial flow to the viscous force. For a CIJ device, the collective Re numbers is calculated by accumulating the contribution of multiple streams:

$$Re = {\sum }_{i = 1}^{n} {Re}_{i} = \frac{4}{π d} {\sum }_{i = 1}^{n} \frac{{ρ}_{i} {Q}_{i}}{{μ}_{i}} \qquad ( 1 )$$

whereas ρᵢ is the density of the solution in the ith inlet stream (kg/m³); Qᵢ is the flow rate of the ith inlet stream (m³/s); μᵢ is the fluid viscosity of the ith inlet stream (Pa s); dᵢ is the diameter of the ith inlet nozzle (m) and n is the number of streams.

In some embodiments, the Reynolds number achieved during mixing of the reactants is about 1,000 to about 20,000, such as about 1,600 to about 10,000, about 2,000 to about 10,000, about 2,000 to about 8,000, about 1,900 to about 5,000, and about 3,000 to about 5,000.

In some embodiments, the variable flow rates of the streams range from about 1 milliliter (mL)/minute to about 50 mL/minute, such as between about 3 mL/minute to about 50 mL/minute, such as between about 5 mL/minute to about 30 mL/minute, and between about 10 mL/minute to about 20 mL/minute. In some embodiments, the variable flow rates of the streams are greater than about 10 mL/minute. In other embodiments, the variable flow rates of the streams are greater than about 3 mL/minute.

In particular embodiments, the first variable flow rate, the second variable flow rate, and the third variable flow rate, if present, are each equal to or greater than about 10 milliliters/minute (mL/min). In yet more particular embodiments, the first variable flow rate, the second variable flow rate, and the third variable flow rate, if present, are each between about 10 mL/min to about 20 mL/min, including 10, 11, 12, 13, 14, 15, 16, 17, 18, 19, and 20 mL/min.

In certain embodiments, the characteristic mixing time is between about 1 ms to about 200 ms, including between about 1 ms to about 100 ms, and between about 1 ms to about 25 ms. In some embodiments, the characteristic mixing time is shorter than about 20 ms. In certain embodiments, the characteristic mixing time is between about 1 ms to about 25 ms, including about 1, 10, 15, 20, and 25 ms. In particular embodiments, the characteristic mixing time is about 15 ms.

In some embodiments, the ratio of the flow rate of the second stream to the flow rate of the first stream is from about 0.1 to about 10.

In some embodiments, an additive is included within a stream. For example, a therapeutic agent may be added to either a stream containing a water-soluble polycation and/or a second stream containing a water-soluble polyanion. In some embodiments, the first stream and/or the second stream further comprise one or more water-soluble therapeutic agents. In some embodiments, the generated PEC nanoparticles encapsulate at least one or more water-soluble therapeutic agents.

In some embodiments, one or more water-soluble therapeutic agents are selected from the group consisting of small molecules, such as small organic or inorganic molecules; saccharides; oligosaccharides; polysaccharides; a biological macromolecule selected from the group consisting of peptides, proteins, peptide analogs and derivatives; peptidomimetics; nucleic acids, such as DNA, RNA interference molecules, selected from the group consisting of siRNAs, shRNAs, antisense RNAs, miRNAs and ribozymes, dendrimers and aptamers; antibodies, including antibody fragments and intrabodies; an extract made from biological materials selected from the group consisting of bacteria, plants, fungi, animal cells, and animal tissues; naturally occurring or synthetic compositions; and any combination thereof. In some embodiments, one or more water-soluble therapeutic agents are selected from the group consisting of a small molecule, carbohydrate, sugar, protein, peptide, nucleic acid, antibody or antibody fragment thereof, hormone, hormone receptor, receptor ligand, cytokine, and growth factor.

In some embodiments, one or more water-soluble polycationic polymers are selected from the group consisting of chitosan, PAMAM dendrimers, polyethylenimine (PEI), protamine, poly(arginine), poly(lysine), poly(beta-aminoesters), cationic peptides and derivatives thereof.

In some embodiments, one or more water-soluble polyanionic polymers are selected from the group consisting of poly(aspartic acid), poly(glutamic acid), negatively charged block copolymers (poly(ethylene glycol)-b-poly(acrylic acid), poly(ethylene glycol)-b-Poly(aspartic acid), poly(ethylene glycol)-b-poly(glutamic acid), heparin sulfate, dextran sulfate, hyaluronic acid, alginate, tripolyphosphate (TPP), poly(glutamic acid), a cytokine (e.g., a chemokine, interferon, interleukin, lymphokine, tumor necrosis factor), a protein, a peptide, a growth factor, and a nucleic acid.

The terms “polypeptide” and “protein” as used herein refer to a polymer of amino acids. As used herein, a “peptide” refers to short chain of amino acid monomers, such as about 50 or fewer amino acids.

As used herein, a “growth factor” refers to a substance, such as a protein or hormone, which is capable of stimulating cellular growth, proliferation, healing, and/or cellular differentiation. Non-limiting examples of growth factors include platelet derived growth factor (PDGF), transforming growth factor β (TGF-β), insulin-related growth factor-I (IGF-I), insulin-related growth factor-II (IGF-II), fibroblast growth factor (FGF), beta-2-microglobulin (BDGF II), and bone morphogenetic factors.

As used herein, a “nucleic acid” or “polynucleotide” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes.

In some embodiments, the nucleic acid is an RNA interfering agent. As used herein, an “RNA interfering agent” is defined as any agent that interferes with or inhibits expression of a target gene, e.g., by RNA interference (RNAi). Such RNA interfering agents include, but are not limited to, antisense molecules, ribozymes, small inhibitory nucleic acid sequences, for example but not limited to guide RNAs, small interfering RNA (siRNA), short hairpin RNA or small hairpin RNA (shRNA), microRNA (miRNA), post-transcriptional gene silencing RNA (ptgsRNA), short interfering oligonucleotides, antisense oligonucleotides, aptamers, CRISPR RNAs, nucleic acid molecules including RNA molecules which are homologous to the target gene, or a fragment thereof, and any molecule which interferes with or inhibits expression of a target gene by RNA interference (RNAi).

In some embodiments, the nucleic acid is selected from the group consisting of an antisense oligonucleotide, cDNA, genomic DNA, guide RNA, plasmid DNA, vector DNA, mRNA, miRNA, piRNA, shRNA, and siRNA. In some embodiments, the nucleic acid is not siRNA. As used herein, the term “plasmid DNA” refers to a small DNA molecule that is typically circular and is capable of replicating independently.

In some embodiments, the plasmid DNA concentration is between about 25 to about 800 μg/mL, including 25, 50, 100, 200, 300, 400, 500, 600, 700, and 800 μg/mL. In particular embodiments, the plasmid concentration is selected from the group consisting of about 25 μg/mL, about 50 μg/mL, about 100 μg/mL, about 200 μg/mL, about 400 μg/mL, and about 800 μg/mL.

In some embodiments, one or more water-soluble polyanionic polymers is plasmid DNA and one or more water-soluble polycationic polymers is selected from the group consisting of linear polyethylenimine (PEI) and its derivatives, such as but not limited to, poly(ethylene glycol)-b-PEI and poly(ethylene glycol)-g-PEI.

In some embodiments, the second stream comprises one or more water-soluble therapeutic agents and the polyelectrolyte complexation process encapsulates one or more water-soluble therapeutic agents in the generated polyelectrolyte complex (PEC) nanoparticles.

In some embodiments, the polyelectrolyte complex nanoparticles comprise polycations and polyanions such as chitosan/TPP, protamine/heparin sulfate, PEI/DNA, chitosan-g-PEG17/Glu5, chitosan/poly-aspartic acid sodium salt and protamine sulfate/heparin. In some embodiments, the first stream comprises chitosan and the second stream comprises tripolyphosphate (TPP) and a protein, wherein the protein is co-encapsulated by the TPP and chitosan in the generated polyelectrolyte complex (PEC) nanoparticles. The concentrations of the polycation and polyanion will depend upon the specific macromolecules used and the desired shape and uniformity of the resulting polyelectrolyte complex nanoparticles. Specific embodiments are described in the Examples below.

In some embodiments, increasing the concentration of the water-soluble polycation and/or water-soluble polyanion and/or increasing the pH of a stream can affect the shape, particle size and/or particle size uniformity. For example, when the concentration of a stream containing DNA is increased, while the concentration of a water-soluble polycation such as PEI remains constant, the shape of the resultant nanoparticles formed during flash nanocomplexation may, in some embodiments, be generally more rod-like rather than spherical. Further, increasing the pH of either the water-soluble polycation and/or water-soluble polyanionic streams can also result in more rod-like shaped nanoparticles. Conversely, spherically shaped nanoparticles may generally be obtained in some embodiments, by increasing the concentration and/or pH of the water-soluble polycation and/or water-soluble polyanion streams.

As described herein, in some embodiments, the stream will contain an additive such as a therapeutic agent, for example, a water-soluble therapeutic agent. For example, a water-soluble therapeutic agent, such as a protein, may be added to a stream containing a water-soluble polyanion, such as TPP. The water-soluble polyanionic stream containing the protein and the water-soluble polycationic stream containing chitosan, for example, may be independently loaded into a syringe of a CIJ device to obtain protein-containing nanoparticles co-encapsulated by the chitosan and the TPP.

In some embodiments, a water-soluble therapeutic agent such as a nucleic acid, e.g. siRNA, may complexed with, for example, a water-soluble polycation such as PEI in a nanoparticle. Accordingly, a water-soluble polyanion of the present disclosure may be used to both form the instant polyelectrolyte complex nanoparticle described herein and to act as a therapeutic agent.

### II. Polyelectrolyte Complex Nanoparticles

In some embodiments, the presently disclosed subject matter provides a uniform polyelectrolyte complex (PEC) nanoparticle preparation generated from a flash nanocomplexation (FNC) method, the method comprising: (a) flowing a first stream comprising one or more water-soluble polycationic polymers at a first variable flow rate into a confined chamber; (b) flowing a second stream comprising one or more water-soluble polyanionic polymers at a second variable flow rate into the confined chamber, wherein the first stream and the second stream are on opposing sides when entering the confined chamber; and (c) optionally flowing a third stream comprising one or more components selected from the group consisting of one or more water-soluble therapeutic agents, one or more miscible organic solvents, and/or one or more cryoprotectants at a third variable flow rate into the confined chamber; wherein each stream is equidistant from the other two streams when entering the confined chamber; wherein the first variable flow rate, the second variable flow rate, and the third variable flow rate, if present, can be the same or different; and (d) impinging the first stream, the second stream, and the third stream, if present, in the confined chamber until the Reynolds number is from about 1,000 to about 20,000, thereby causing the one or more water-soluble polycationic polymers and the one or more water-soluble polyanionic polymers to undergo a polyelectrolyte complexation process that continuously generates PEC nanoparticles, wherein the polyelectrolyte complexation process occurs under conditions having a characteristic assembly time (TA), over which assembly of the PEC nanoparticles occurs, which is greater than a characteristic mixing time (τ_{M}), over which components of the first stream, second stream, and third stream, if present, are mixed homogenously.

The presently disclosed uniform polyelectrolyte complex nanoparticles have particle sizes, distributions of particle sizes, and polyanion and polycation components as described above and in the Examples below. In some embodiments, the uniform polyelectrolyte complex nanoparticles of the present disclosure encapsulate one or more additives, as described herein, such as water-soluble therapeutic agents.

In some embodiments, the polyelectrolyte complex nanoparticles, which are formed according to the present methods are uniform in particle size, i.e., there is a narrow distribution of particle size. For example, in some embodiments, the present nanoparticles have an average particle size of less than about 500 nm, less than about 100 nm, less than about 60 nm, or less than about 40 nm (homogenous diameter). In some embodiments, the generated polyelectrolyte complex nanoparticles range in size from about 20 nm to about 500 nm in diameter. In some embodiments, the generated polyelectrolyte complex nanoparticles range in size from about 25 nm to about 100 nm in diameter. In some embodiments, the generated polyelectrolyte complex nanoparticles range in size from about 30 nm to about 80 nm in diameter. In some embodiments, the generated polyelectrolyte complex nanoparticles range in size from about 25 nm to about 60 nm in diameter. In some embodiments, the generated polyelectrolyte complex nanoparticles range in size from about 30 nm to about 45 nm in diameter. In some embodiments, the generated polyelectrolyte complex nanoparticles are about 30 nm in diameter. In particular embodiments, the generated polyelectrolyte complex nanoparticles range in size from about 30 nm to about 80 nm in diameter. In yet more particular embodiments, the nanoparticle has an average size between about 35 nm to about 130 nm, including 35, 40, 45, 50, 55, 60, 65, 70, 75, 80, 85, 90, 100, 105, 110, 115, 120, 125, and 130 nm. In particular embodiments, the PEC nanoparticle has an average size of about 80 nm.

In some embodiments, the presently disclosed PEC nanoparticle has an average of about 1 to about 50 copies of pDNA per nanoparticle, including 1, 1.5, 2.0, 2.5, 3, 3.5, 4, 4.5, 5, 5.5, 6, 6.5. 7, 7.5, 8, 8.5, 9, 9.5, 10, 15, 20, 25, 30, 35, 40, 45, and 50 copies of pDNA per nanoparticle. In particular embodiments, the PEC nanoparticle has an average of about 1.3 to about 21.8 copies of pDNA per nanoparticle; about 1.3 to about 1.4 copies of pDNA per nanoparticle; about 1.3 to about 1.6 copies of pDNA per nanoparticle; about 1.3 to about 1.7 copies of pDNA per nanoparticle; about 1.3 to about 2.3 copies of pDNA per nanoparticle; about 1.3 to about 2.6 copies of pDNA per nanoparticle; about 1.3 to about 3.5 copies of pDNA per nanoparticle; about 1.3 to about 4.4 copies of pDNA per nanoparticle; about 1.3 to about 4.7 copies of pDNA per nanoparticle; about 1.3 to about 5.0 copies of pDNA per nanoparticle; about 1.3 to about 6.1 copies of pDNA per nanoparticle; about 1.3 to about 8.0 copies of pDNA per nanoparticle; about 1.3 to about 8.5 copies of pDNA per nanoparticle; about 1.3 to about 9.1 copies of pDNA per nanoparticle; about 1.3 to about 9.5 copies of pDNA per nanoparticle; about 1.3 copies of pDNA per nanoparticle; about 3.5 copies of pDNA per nanoparticle; about 4.4 copies of pDNA per nanoparticle; about 5.0 copies of pDNA per nanoparticle; about 6.1 copies of pDNA per nanoparticle; about 8.0 copies of pDNA per nanoparticle; about 8.1 copies of pDNA per nanoparticle; about 8.5 copies of pDNA per nanoparticle; about 9.1 copies of pDNA per nanoparticle; about 9.5 copies of pDNA per nanoparticle; about 1.3 to about 10.0 copies of pDNA per nanoparticle; about 1.3 to about 13.5 copies of pDNA per nanoparticle; or about 21.8 pDNA copies per nanoparticle. In particular embodiments, the PEC nanoparticle has an average of less than 40 copies of pDNA per nanoparticle.

In particular embodiments, the PEC nanoparticle has an average of about 1.3 to about 21.8 copies of pDNA per nanoparticle; in some embodiments, between about 1.3 to about 13.5 copies of pDNA per nanoparticle, including 1.3, 1.7, 2.3, 4.7, and 13.5 copies of pDNA per nanoparticle, for example for I2 plasmid; in some embodiments, between about 1.6 to about 10.0 copies of pDNA per nanoparticle, including 1.6, 1.7, 2.6, 6.1, and 10.0 copies of pDNA per nanoparticle, for example, for gWiz-GFP; in some embodiments, about 1.4 to about 21.8 copies of pDNA per nanoparticle, including 1.4, 1.7, 3.5, 6.1, and 21.8 copies of pDNA per nanoparticle, for example for gWiz-Luc; and, in some embodiments, between about 4.4 to about 9.1 copies of pDNA per nanoparticle, including 4.4, 5.0, 6.1, and 9.1 copies of pDNA per nanoparticle copies of pDNA per nanoparticle, for example for gWiz-Luc with varying N/P ratios. In yet more particular embodiments, the PEC nanoparticle has one pDNA per nanoparticle.

In some embodiments, the PEC nanoparticle comprises polyethylenimine and plasmid DNA. In certain embodiments, the PEC nanoparticle has a ratio of amine in the polyethylenimine to phosphate in the plasmid DNA (N/P) between about 3 to about 6. In particular embodiments, the PEC nanoparticle has an N/P selected from the group consisting of about 3, about 4, about 5, and about 6. In yet more particular embodiments, the PEC nanoparticle has a percentage of bound WEI to total WEI between about 50% to about 75%, including about 50, 55, 60, 65, 70, 71, 72, 73, 74, and 75% bound WEI to total WEI.

In some embodiments, the polydispersity index (PDI) of a plurality of the generated polyelectrolyte complex nanoparticles may range from about 0.05 to about 0.2. In particular embodiments, the plurality of PEC nanoparticles has a PDI between about 0.1 and about 0.25, including 0.01, 0.11, 0.12, 0.13, 0.14, 0.15, 0.16. 0.17, 0.18, 0.19, 0.20, 0.21, 0.22, 0.23, 0.24, and 0.25.

In some embodiments, the PEC nanoparticle has a surface charge between about +20 to about +50 mV, including +20, +21. +22. +23, +24, +25, +26, +27, +28, +29, +30, +31, +32, +33, +34, +35, +36, +37, +38, +39, +40, +41, +42, +43, +44, +45, +46, +47, +48, +49, and +50 mV. In some embodiments, the PEC nanoparticle has an apparent hydrodynamic density between about 60 Da/nm³ to about 80 Da/nm³, including 60, 61, 62, 63, 64, 65, 66, 67, 68, 69, 70, 71, 72, 73, 74, 75, 76, 77, 78, 79, and 80 Da/nm³. In particular embodiments, the PEC nanoparticle has an apparent hydrodynamic density of about 67.68 Da/nm³.

In some embodiments, the presently disclosed subject matter provides a pharmaceutical formulation comprising the presently disclosed PEC nanoparticle or plurality of PEC nanoparticles in a pharmaceutically acceptable carrier. In certain embodiments, the pharmaceutical formulation comprises a lyophilized formulation. In particular embodiments, the pharmaceutical formulation of the PEC nanoparticle or plurality of PEC nanoparticles exhibits long term stability at −20° C. for at least 9 months, including 1, 2, 3, 4, 5, 6, 7, 8, and 9 months.

As used herein, “pharmaceutically acceptable carrier” is intended to include, but is not limited to, water, saline, dextrose solutions, human serum albumin, liposomes, hydrogels, microparticles and nanoparticles. The use of such media and agents for pharmaceutically active compositions is well known in the art, and thus further examples and methods of incorporating each into compositions at effective levels need not be discussed here.

Depending on the specific conditions being treated, the presently disclosed nanoparticles may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20th ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery.

While the form and/or route of administration can vary, in some embodiments the presently disclosed nanoparticles or pharmaceutical composition is administered parenterally (e.g., by subcutaneous, intravenous, or intramuscular administration), or in some embodiments is administered directly to the lungs. Local administration to the lungs can be achieved using a variety of formulation strategies including pharmaceutical aerosols, which may be solution aerosols or powder aerosols. Powder formulations typically comprise small particles. Suitable particles can be prepared using any means known in the art, for example, by grinding in an air jet mill, ball mill or vibrator mill, sieving, microprecipitation, spray-drying, lyophilization or controlled crystallization. Typically, particles will be about 10 microns or less in diameter. Powder formulations may optionally contain at least one particulate pharmaceutically acceptable carrier known to those of skill in the art. Examples of suitable pharmaceutical carriers include, but are not limited to, saccharides, including monosaccharides, disaccharides, polysaccharides and sugar alcohols such as arabinose, glucose, fructose, ribose, mannose, sucrose, trehalose, lactose, maltose, starches, dextran, mannitol or sorbitol. Alternatively, solution aerosols may be prepared using any means known to those of skill in the art, for example, an aerosol vial provided with a valve adapted to deliver a metered dose of the composition. Where the inhalable form of the active ingredient is a nebulizable aqueous, organic or aqueous/organic dispersion, the inhalation device may be a nebulizer, for example a conventional pneumatic nebulizer such as an air jet nebulizer, or an ultrasonic nebulizer, which may contain, for example, from 1 mL to 50 mL, commonly 1 mL to 10 mL, of the dispersion; or a hand-held nebulizer which allows smaller nebulized volumes, e.g., 10 μL to 100 μL.

For injection, the agents of the disclosure may be formulated and diluted in aqueous solutions, such as in physiologically compatible buffers such as Hank's solution, Ringer's solution, or physiological saline buffer or an isotonic sugar solution.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances such as, saline, preservatives, such as benzyl alcohol, absorption promoters, and fluorocarbons.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

### Example 1

### Kinetic Control in Assembly of Plasmid DNA/Polycation Complex Nanoparticles

1.1 Overview. Polyelectrolyte complex (PEC) nanoparticles assembled from plasmid DNA (pDNA) and polycations, such as linear polyethyleneimine (lPEI), represent a major non-viral delivery vehicle for gene therapy. Efforts to control the size, shape and surface properties of pDNA/polycation nanoparticles have primarily focused on fine-tuning molecular structures of the polycationic carriers and assembly conditions, such as medium polarity, pH, and temperature. Reproducible production of these nanoparticles, however, hinges on the ability to control the assembly kinetics, given the non-equilibrium nature of the assembly process and nanoparticle structure.

In some embodiments, the presently disclosed subject matter adopts a kinetically controlled mixing process, referred to herein as “flash nanocomplexation” or “(FNC),” to accelerate the mixing of the pDNA solution with the polycation lPEI solution to match the PEC assembly kinetics through turbulent mixing in a microchamber, thus achieving explicit control of the kinetic conditions for pDNA/lPEI nanoparticle assembly as demonstrated by the tunability of nanoparticle size, composition, and pDNA payload. Using a combined experimental and simulation approach, pDNA/lPEI nanoparticles having an average of about 1.7 to about 21.8 copies of pDNA per nanoparticle and average size of about 35 nm to about 130 nm were prepared in a more uniform and scalable manner than bulk mixing methods. Using these nanoparticles with well-defined compositions and sizes, pDNA payload and nanoparticle formulation composition could be correlated with transfection efficiencies and toxicity of these nanoparticles. These nanoparticles exhibited long term stability at −20° C. for at least 9 months in a lyophilized formulation, validating scalable manufacture of an off-the-shelf nanoparticle product with well-defined characteristics for gene therapy.

1.2 Background The polyelectrolyte nature of the assembly components indicates slower diffusion rates of the polymer chains compared to their electrostatic complexation rate. As a result, the PEC assembly yields non-equilibrium, kinetically arrested complex structures. During the assembly process, the transient and local concentration profiles of different components determine how each PEC assembly initiates, propagates, and terminates to form a distinct nanoparticle. Control over these kinetic conditions is only possible when mixing is faster than the assembly process to allow distribution of the assembly components in a homogenous manner before nanoparticle starts to assemble. Homogeneous mixing not only ensures that PEC nanoparticles are produced with uniform characteristics, but also provides opportunity to control the size, surface properties, and compositions of the nanoparticles through manipulations of the input conditions into the assembly system. This assembly condition requires that the characteristic mixing time (τ_{M}), over which different assembly components are mixed homogenously, is reduced to less than the characteristic assembly time (τ_{A}), over which the PEC nanoparticle assembly occurs. Conventional mixing methods including pipetting and vortexing cannot fulfill this requirement.

Mixing occurs through diffusion of assembly components across the interfaces of different flows. The most common approach to achieve small τ_{M} is by shortening the diffusion path, which can be achieved by both laminar flow and turbulent flow set-ups. In a laminar flow set-up, mixing is achieved as different flow paths are introduced within a small compartment. Due to manufacturing difficulties, however, engineering approaches, such as hydrodynamic focusing, Lu et al., 2014; Lu et al., 2016, and droplet confinement, Juul et al., 2012, are developed to further increase surface-to-volume ratio. In contrast, in a turbulent flow set-up, turbulent eddies enable rapid flow breakdown to tiny dimensions for efficient diffusion. Flow turbulence can be delivered by “T” connectors, Kasper et al., 2011, Tesla mixers and herring-bone mixers, Feng et al., 2016, coaxial jet mixers, Liu et al., 2015; Liu et al., 2017, confined impinging jets (CIJ), Johnson and Prud'homme; Liu and Fox, 2006, and multi-inlet vortex mixers (MIVM). Liu et al., 2008; He et al., 2017; He et al., 2018.

Various degrees of success have been achieved in preparing drug-loaded nanoparticles with more uniform characteristics compared with conventional methods, owing to a higher degree of control of mixing kinetics of the assembly components. Turbulent mixing in a CIJ mixer was recently adopted to generate pDNA/lPEI nanoparticles to demonstrate the scalability of the method and feasibility to control the size, Santos et al., 2016, but the kinetics of mixing and nanoparticle assembly have not been analyzed. Kinetically restricted assembly has been well illustrated for self-assembly of amphiphilic polymeric micelles where nanoparticle formation can be tuned by solvent mixing rate vs. polymer aggregation and drug partition rates in mixed solvents in a process called flash nanoprecipitation (FNP). Saad and Prud'homme, 2016. FNP uses turbulent mixing in an MIVM or CIJ mixer to mix two opposing jets carrying miscible solvents in a time shorter than characteristic time for hydrophobic chain aggregation. Uniform nanoparticles can be produced as a result of homogenous supersaturation conditions. Nikoubashman et al., 2016; Zhang et al., 2012. By varying kinetic conditions under such mixing status, a diffusion-limited and fusion-dominated aggregation mechanism for nanoparticle formation, Johnson and Prud'homme, 2003, and a quantitative model for predicting nanoparticle size, Pagels et al., 2018, have been proposed.

1.3 Scope of Work. The presently disclosed subject matter, in part, investigates the kinetic control aspects of pDNA/polycation PEC nanoparticle assembly. More particularly, the presently disclosed subject matter demonstrates the kinetic control of PEC assembly and nanoparticle formation using a turbulent mixing approach in a CIJ mixer termed “flash nanocomplexation (FNC).” The diffusion kinetics of polyelectrolytes pDNA and linear polyethyleneimine (lPEI) in FNC is significantly different from that of solvent and polymer in FNP, where the complexation kinetics mediated by polyelectrolyte charge neutralization is faster than hydrophobic aggregation of the polymer chain segments in FNP, and the PEC occurs in aqueous medium absent of organic solvent mixing that occurs in FNP. These factors contribute to the unique process and additional challenges for kinetic control of PEC assembly into nanoparticles in FNC. For the presently disclosed study, in vivo-jetPEI® was selected as the testing carrier due to its high transfection efficiency in vivo as the benchmark for non-viral carriers, its availability in GMP quality, and its molecular simplicity as a polycation with uniform charge density. Using PEC nanoparticles of in vivo-jetPEI® and plasmids of typical sizes of 4 kb to 7 kb as a model system, the mixing flow regimen in a CIJ mixer was examined using fluid dynamic simulations and the requirements of achieving kinetic control over the PEC assembly process were analyzed. Exquisite control over the pDNA/in vivo-jetPEI® nanoparticle composition was demonstrated through manipulation of kinetic conditions, and the effect of nanoparticle composition, size and surface characteristics on their transfection efficiency in vitro and in vivo was characterized. The advantages of pDNA/in vivo-jetPEI® nanoparticles assembled under kinetically controlled conditions was analyzed in terms of their transfection efficiency and translational potentials for non-viral gene therapy.

1.4 Representative Results and Discussion. Mixing occurs as molecular diffusion across the interfaces between the pDNA and lPEI flows in the turbulent structures.

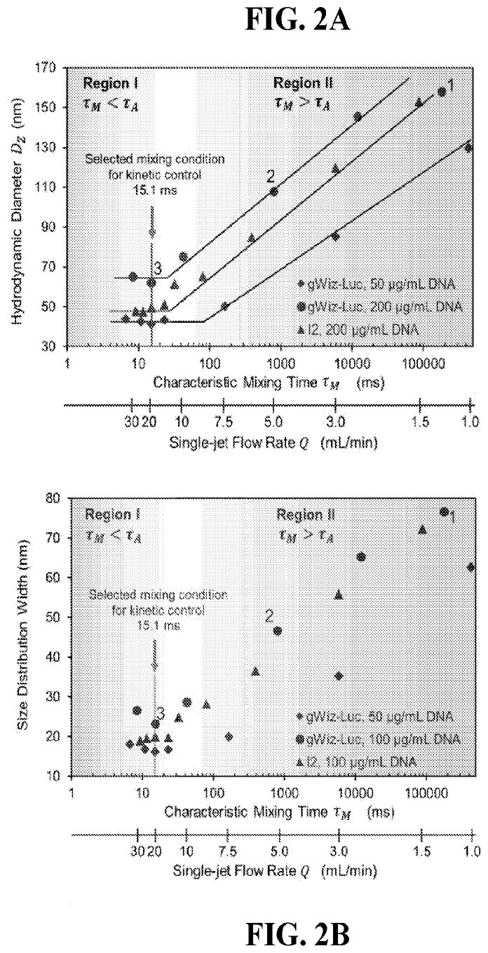

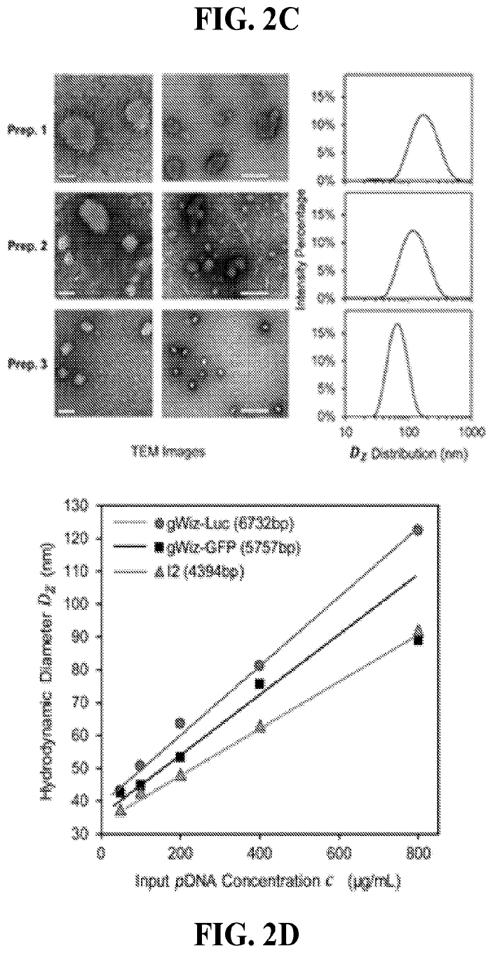

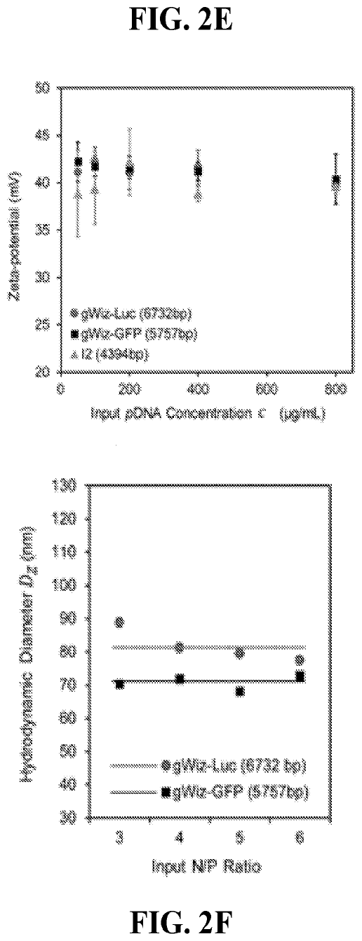

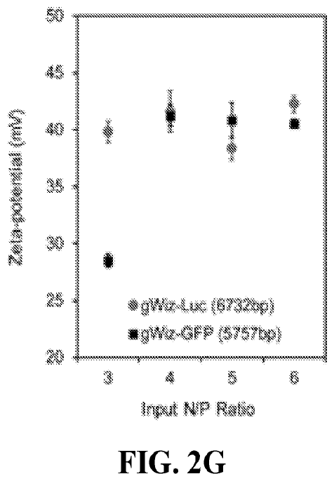

1.4.1. Effect of characteristic mixing time τ_{M} on the outcomes for pDNA/lPEI nanoparticle assembly. Next, the effect of flow rate Q on FNC assembly of pDNA/lPEI nanoparticles when impinging a gWiz-LucpDNA (6.7 kb) solution was experimentally examined. The following conditions were not varied in this study: the pH of the WET solution was kept consistent at 3.5 to maintain the same protonation degree of lPEI around 75%, and thus the same charge density for WEI. Curtis et al., 2016. Similarly, the concentration of the DNA solution was maintained at examined at a concentration of 200 μg/mL and the in vivo-jetPEI® solution at a concentration corresponding to an N/P of 4. When increasing Q, i.e., decreasing τ_{M}, the size (z-average hydrodynamic diameter, Dg) given by dynamic light scattering (DLS) measurement of the nanoparticles decreased until it reached a plateau of a lower limit (FIG. 2A).

When plasmid concentration was decreased from 200 μg/mL to 50 μg/mL, or the plasmid size reduced from 6.7 kb to 4.4 kb (I2 plasmid, see Table 1), the measured nanoparticle size followed the same trend. The critical Q₀ above which consistent DLS size was obtained was lowered from approximately 15 mL/min to approximately 8.5 mL/min (critical τ_{M,0} increased from approximately 20 ms to approximately 85 ms) by the lowered input pDNA concentration. The size distribution width (see Section 1.6) of the nanoparticles given by DLS showed the same dependency on Q (FIG. 2B), indicating increased uniformity of the nanoparticles as τ_{M} decreases. The flow rate-dependent average size and uniformity were confirmed by transmission electron microscopy (TEM) observations (FIG. 2C).

TABLE 1
Representative plasmid DNAs used in this study

| Plasmid | Length | Description |
|---|---|---|
| gWiz-Luc | 6732 bps | Encodes luciferase protein as a reporter. In this study, serves mainly for |
| | | the purpose of evaluation of transfection efficiency in vitro or in vivo. |
| gWiz-GFP | 5757 bps | Encodes GFP protein. In this study, serves only for the purpose of |
| | | varying plasmid size. |
| I2 | 4393 bps | Proprietary plasmid construct provided by Cancer Targeting Systems |
| | | Inc. In this study, serves only for the purpose of varying plasmid size. |
| PEG-Luc | 5314 bps | Constructed with PEG-3 promoter, thus providing tumor-specific |
| | | transfection and expression of luciferase protein as a reporter. |

Based on these findings, the field of characteristic mixing time τ_{M} or flow rate Q could be divided into two regions:

“Region I” corresponds to the kinetic condition where the average DLS size and uniformity remained constant independent of Q or τ_{M}. This indicates that the mixing conditions within the microchamber has reached the maximum degree of homogeneity to allow the assembly to occur uniformly, so that all nanoparticles have a similar assembly path. This assembly process has a time scale defined as the characteristic assembly time (τ_{A}), and with τ_{M}<τ_{A}, almost all pDNA/lPEI nanoparticles are assembled under the same defined conditions (concentrations of pDNA and lPEI, temperature, medium pH, ionic strength, and the like). In other words, the assembly components pDNA and lPEI can be mixed at a rate that is faster than nanoparticle formation to initiate nanoparticle assembly nearly “simultaneously” and in nearly the same microenvironment. As discussed hereinabove, D_{PEI}>>D_{DNA}, and it is lPEI molecules that primarily diffuse into pDNA flow regions, resulting in homogeneous distribution of lPEI molecules to the vicinity of pDNA molecules. This establishes uniform initial kinetic conditions defined by the input concentration profiles of pDNA and lPEI.

“Region II” corresponds to the kinetic condition where τ_{M}>τ_{A} such that the molecular mixing process occurs on a time scale that is greater than the nanoparticle assembly process. Under this condition, nanoparticle assembly happens in a heterogeneous manner as mixing progresses, and partially formed nanoparticles can further associate with late-arrival molecules in an undefined manner. This leads to nonuniform and likely larger nanoparticle size; and compositions of the nanoparticles are dependent on the flow rate and mixing condition. As the flow rate increases and approaches the critical condition allowing τ_{M}=τ_{A}, the assembly mixture is more closer to turbulent mixing structure, and becoming more homogenous; the mixture composition becomes closer to the input concentration profiles of pDNA and lPEI. For example, when comparing Preparation 1 (Q=1.25 mL/min and τ_{M}=1.8×10⁵ ms), Preparation 2 (Q=5 mL/min and τ_{M}=7.9×10² ms), and Preparation 3 (Q=20 mL/min and τ_{M}=15 ms), the nanoparticle ensembles showed the characteristics consistent with this analysis (FIG. 2A, FIG. 2C). As the flow rate increases, i.e., as τ_{M} decreases, the flow mixing profile undergoes a transition from the laminar to turbulent mixing, which coincides with the nanoparticle assembly transition from Region II (τ_{M}>τ_{A}) to Region I (τ_{M}<τ_{A}). This shows the capability of turbulent mixing in a CIJ device to match the solution mixing time scale with the pDNA/WEI nanoparticle assembly time scale by varying the flow rates from 1 mL/min to 50 mL/min.

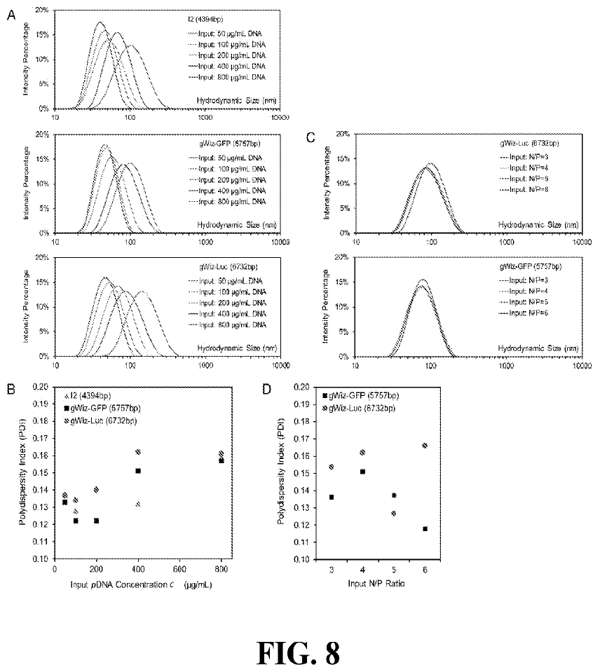

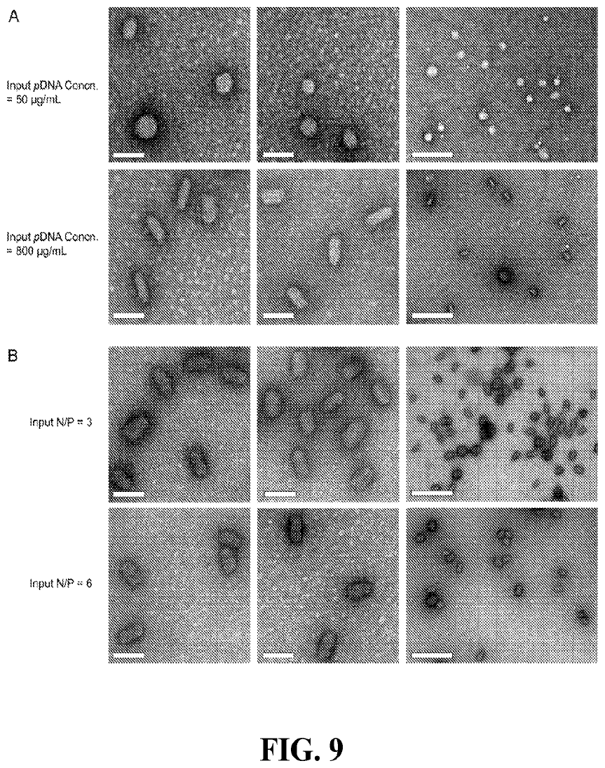

1.4.2. Effect of pDNA concentration and N/P ratio on pDNA/lPEI nanoparticle assembly. When assembly occurs under the kinetic conditions defined in Region I, the assembly concentration profiles of pDNA and lPEI are well defined by the input concentration profiles (i.e., pDNA concentrations and NIP ratio in the impinging solutions). This provides an opportunity to examine the effect of assembly concentration profiles on nanoparticle characteristics. A flow rate of 20 mL/min (τ_{M}=15 ms, as labeled in FIG. 2A, FIG. 2B) was selected for this comparison. As shown in FIG. 2D, an increase in pDNA concentration resulted in increase of nanoparticle size. Relatively narrow size distribution and low PDI (0.12-0.16) of these formulations (FIG. 8A, FIG. 8B); and TEM observations (FIG. 9A) confirmed the kinetically controlled mixing and uniform assembly of the pDNA/lPEI nanoparticles. On the other hand, when input pDNA concentration was fixed at 400 μg/mL, nanoparticles assembled at different N/P ratios gave similar size (FIG. 2E and FIG. 9B), indicating that the plasmid was compacted most effectively under this mixing condition independent of the initial lPEI concentration, and to a maximum compaction degree even when the N/P ratio was reduced to 3. This observation further confirms the effectiveness of the turbulent mixing with τ_{M}<τ_{A} in maximizing the access of lPEI molecules to complex with pDNA during the assembly process. In addition, zeta-potential assessments revealed a similar surface charge around +40 mV for all formulations regardless of the plasmid used, pDNA concentration (FIG. 2E) or input N/P ratio (FIG. 2G). This suggests that the nanoparticle surfaces are similar and consist of excess amount of lPEI molecules. Conventional mixing methods for preparation of pDNA/polycation nanoparticles, such as pipetting, provide a mixing time on an order of seconds, therefore falling into Region II on this kinetics scale. The method of pipetting followed by vortexing (Table 2) generated nanoparticles with average sizes and uniformity that are similar to those of FNC preparations with a flow rate Q<1.5 mL/min (FIG. 9). There was no clear dependence of the nanoparticle size on input pDNA concentration. Moreover, there was a higher degree of variability as a result of different pipetting procedures (Table 2) employed in Example 1.4.7.

TABLE 2
Procedures to prepare PEC nanoparticles by pipetting

| Procedure | Description |
|---|---|
| B1 | Slow (2 sec) addition of 50 μL PEI working solution on top of 50 μL DNA working |
| | solution, immediately following vortex for 30 sec; and 5 min of stabilization applied. |
| B2 | Fast injection of 50 μL PEI working solution into 50 μL DNA working solution, |
| | immediately following vortex for 30 sec; and 5 min of stabilization applied. |
| B3 | Fast injection of 100 μL PEI working solution into 100 μL DNA working solution, |
| | immediately following vortex for 30 sec; and 5 min of stabilization applied. |
| B4 | Fast injection of 50 μL DNA working solution into 50 μL PEI working solution, |
| | immediately following vortex for 30 sec; and 5 min of stabilization applied. |

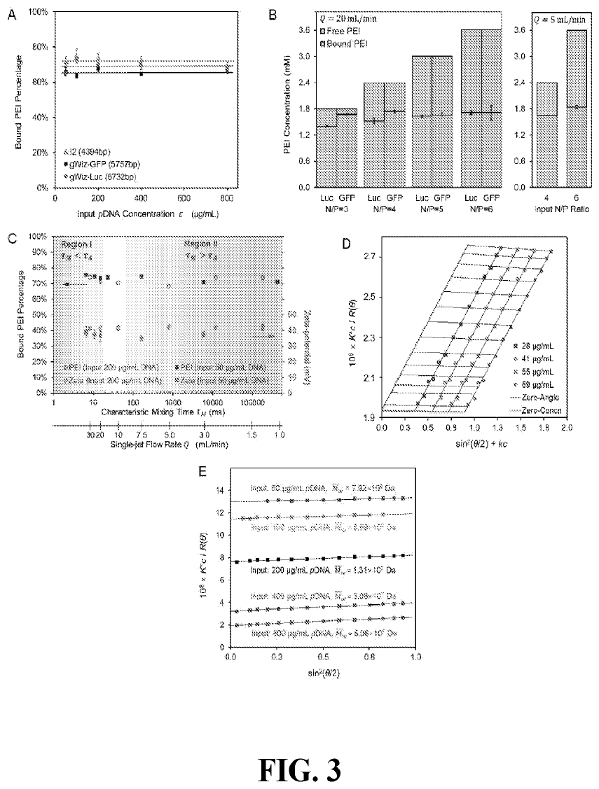

1.4.3. Average nanoparticle composition and free lPEI measurement. Full complexation of pDNA by lPEI is achieved with an N/P ratio greater than 3; therefore, assembly with an N/P≥3 would result in an excess of unbound or free lPEI in the nanoparticle suspension. Yue et al., 2011. To assess the actual composition of the assembled nanoparticles, the amount of free lPEI was first characterized according to a published protocol. Bertschinger et al., 2004. When the pDNA/WEI nanoparticles were assembled under the turbulent mixing condition (Q=20 mL/min and τ_{M}=15 ms) as defined in FIG. 2, all nanoparticle formulations with different pDNA concentration inputs had the same bound vs. free lPEI compositions, as long as the input N/P ratio was fixed at 4 (FIG. 3A). The amount of bound lPEI was nearly all around 70%, which corresponded to an N/P ratio of 2.7 in the nanoparticles. When adjusting the input N/P from 3 to 6 with a consistent pDNA concentration input (FIG. 3B, left panel), a consistent amount of lPEI bound to nanoparticles was found regardless of the input N/P ratio. This indicates that the amount of lPEI bound to pDNA was the same among the nanoparticles prepared under different preparation conditions; and this average composition corresponds to an N/P ratio of 2.74±0.14 (n=28 individual preparations) in the nanoparticles. These resultant “overcharging” nanoparticles are consistent with the fact that not all charged groups are accessible to participate in charge neutralization in the process of PEC formation. Berret, 2005. There was a minor difference between the two plasmids tested in that gWiz-Luc seemed to result in slightly lower amount of bound lPEI as N/P ratio decreased. Nonetheless, the overall conclusion is clear that the binding of lPEI to pDNA to form PEC is not affected by the concentrations of either pDNA or lPEI, nor by the input N/P ratio.

1.4.4. Charge neutralization is not a rate-limiting step for PEC nanoparticle assembly. It was found that this consistent minimal bound N/P ratio for pDNA neutralization also was true for nanoparticles prepared under non-turbulent mixing conditions (FIG. 3B right panel and FIG. 3C). The surface charges (i.e., zeta-potentials) measured for nanoparticles prepared under different mixing conditions also remained the same (FIG. 3C). Since lPEI content in the nanoparticles and zeta-potential are directly related to the charge neutralization and complex formation processes, the findings highlighted in FIG. 3A. FIG. 3B, and FIG. 3C suggest that the charge neutralization and pDNA-lPEI binding are not rate limiting for nanoparticle assembly. In other words, charge neutralization occurs at a rate much faster than condensation and chain folding of the pDNA/lPEI PECs into nanoparticles, i.e., it occurs on a time scale that is much shorter than the total characteristic assembly time τ_{A}. The pDNA/lPEI PEC nanoparticle assembly process achieved under kinetically controlled mixing conditions can now be modeled into two distinct steps, which is in agreement with several literature reports (Barreleiro and Lindman, 2003; Santhiya et al., 2012):

Step 1: Charge neutralization step in which lPEI molecules bind to the pDNA as soon as they diffuse into the vicinity of pDNA molecules. In this study, regardless of input pDNA concentrations or N/P ratios, lPEI complexed with pDNA consistently at an N/P ratio of approximately 2.7. This step forms pDNA/lPEI PECs, and is not rate-limiting;

Step 2: PEC chain assembly, where the neutralized pDNA/lPEI complexes undergo deformation and condensation through folding, Osada et al., 2012; Takeda et al., 2017, that significantly reduces the complex volume, i.e., compaction occurs. This is the rate limiting step, such that the time scale for Step 2 is much larger than that of Step 1. Therefore, the characteristic assembly time τ_{A} is primarily determined by the completion time of Step 2. When the neighboring pDNAs or PECs are close enough to diffuse into each other during the assembly process before the structure is stabilized by repulsions from net positively surface charges, compaction and assembly involving multiple PECs could occur, resulting in multiple pDNAs to be packaged into a single distinct nanoparticle.

1.4.5. Characterization of the average pDNA copy number in each pDNA/lPEI nanoparticle. Given that the presently disclosed nanoparticles can be prepared with a narrow size distribution and consistent composition, the molar mass of the nanoparticles was characterized using the static light scattering (SLS) technique. With a fixed in-nanoparticle pDNA/lPEI mass ratio (FIG. 3A), it was assumed that the refractive increment (dn/dc) value of the nanoparticles is constant and follows the additive rule (see Section 1.6). Dai and Wu, 2012. By measuring the intensity of scattered light to obtain Rayleigh scattering ratio with regard to each scattering angle and each nanoparticle mass concentration, and extrapolating concentration and angle-dependence curves to zero concentration and zero angle on a Zimm plot, the weight average molar mass of nanoparticles, M̅_{w,Nanoparticle}, from which the average copy number of pDNA per nanoparticle (N̅) can be calculated (see Section 1.6). Dubin et al., 2012; Hiemenz and Lodge, 2007.

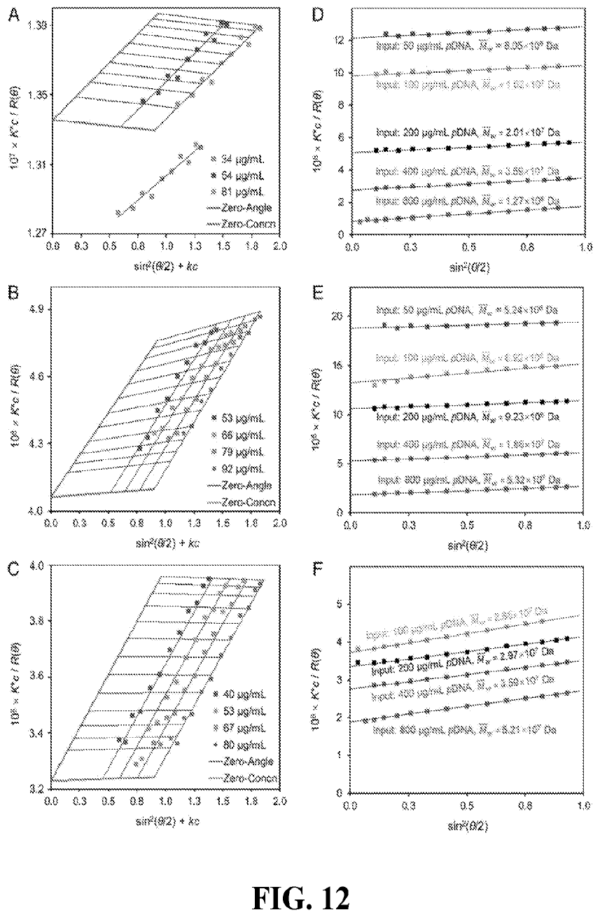

A representative Zimm plot for pDNA/lPEI nanoparticles with an N̅=13.5 is shown in FIG. 3D. For all nanoparticles measured by this method (FIG. 3D and FIG. 12A, FIG. 12B, FIG. 12C), the Zimm plot analyses indicate that the second virial coefficient (A) of these nanoparticles approaches zero. This finding implies that the solvent (water) and temperature (25° C.) conditions used for SLS measurement satisfies the θ condition, i.e., the PEC-solvent interaction cancels out the Vander Waals interaction and volume expansion of the PEC chains such that the PEC chain compaction occurs in a random packing manner. This θ condition significantly simplifies the measurement of average molar mass since the concentration dependence of the light scattering behavior of these nanoparticles can be ignored and the Rayleigh ratios can be measured at a fixed concentration, and calculations using the Debye plots can be measured (FIG. 3E and FIG. 12D, FIG. 12E, FIG. 12F). On the other hand, varying input N/P ratio showed minor changes in the average number of plasmids in each nanoparticle. When input N/P ratio changed from 3 to 6 for input of 400 μg/mL pDNA, N decreased from 9.2 for N/P=3 to 6.1 for N/P=4, 5.0 for N/P=5, and 4.4 for N/P=6 (FIG. 12F).

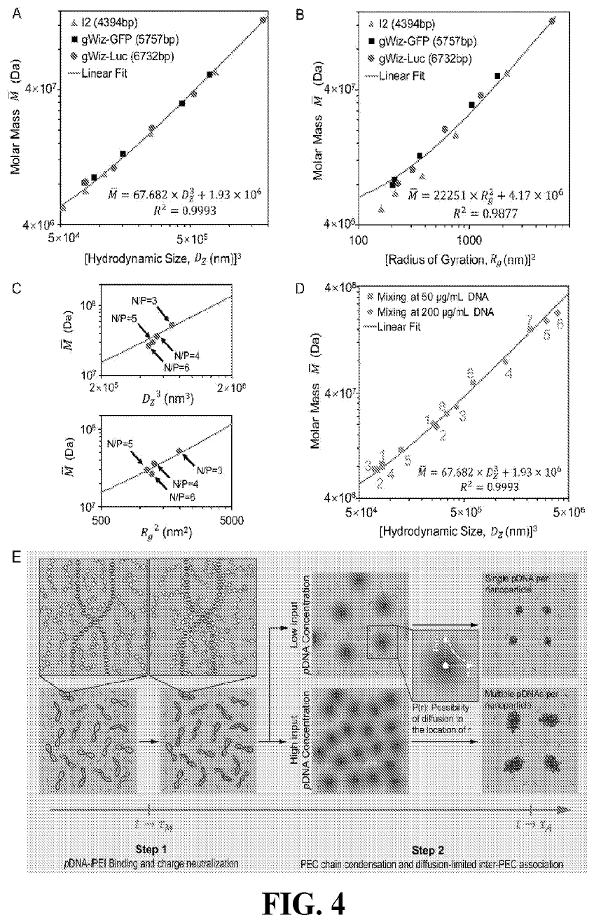

1.4.6. Correlation of DLS size and molar mass of pDNA/lPEI nanoparticles. When the measured weight average molar masses of nanoparticles prepared under different conditions for all three plasmids were plotted against their hydrodynamic volume dimensions (i.e., D_{z}³) (FIG. 4A), a common linear correlation emerged:

[M̅_{w,Nanoparticle},Da]=67.68×[D_{Z},nm]³+1.93×10⁶ (2)

where D_{z} is the z-average size as measured by DLS of the nanoparticle suspension, and M_{w} is the weight average molar mass of the nanoparticles given by SLS. Such a “universal” fit for various nanoparticles independent of the plasmid and conditions used for nanoparticle assembly suggest that the PEC assembly units and compaction degree of these nanoparticles are similar. More specifically, these nanoparticles have the same apparent hydrodynamic density of 67.68 Da/nm³, i.e., pDNAs are condensed to the same degree no matter how many of them are packed into a single nanoparticle.

Similarly, another composition-size correlation that the weight average molar mass of the nanoparticles is linearly proportional to the second power of radius of gyration (i.e. R_{g}²) also was identified (FIG. 4B):

[M̅_{Nanoparticle},Da]=22,251×[R_{g},nm]²+4.17×10⁶ (3)

This correlation fits well for nanoparticles with D_{z} between 50 and 130 nm, and the deviation from experimental data points increases as the size goes smaller than 50 nm. This linear relationship between M̅ and R_{g}² further confirms that these pDNA/lPEI nanoparticles are assembled under the θ condition; and the PEC units assume random packing behavior under the solvent and temperature conditions tested. Hiemenz and Lodge, 2007.

If each PEC chain formed in Step 1 (a pDNA with all its bound lPEI) is considered as a packing unit (i.e., a PEC unit) for nanoparticle assembly in Step 2, a pDNA lPEI nanoparticle can be modeled as an entity comprising either one or multiple PEC units. The nanoparticle assembly follows a quantized combination pattern. Nanoparticles generated by N/Ps varying from 4 to 6 have a similar molar mass, while those generated by N/P=3 are heavier in molar mass but still fall into the two linear fits of Eq. 2 and Eq. 3 (FIG. 4C). It is presumed that with input N/P larger than 2.7, where lPEI is in excess to the amount required to sufficiently compact pDNAs, quantized combination stays valid. This model is further supported by the fact that nanoparticles prepared under laminar mixing condition (τ_{M}>τ_{A}) also follow the same correlation (FIG. 4D). It is remarkable that nanoparticles with lower uniformity (i.e., broader distribution) appeared to have the same apparent hydrodynamic density as that of more uniform nanoparticles with fewer copies of pDNA per nanoparticle. This analysis is consistent with the hypothesis that PEC units formed in Step 1 are the building blocks for nanoparticle assembly, and they are compacted and associated in a similar manner as random folding of PEC unit chains in the solution under the θ condition.

1.4.7. Modeling pDNA/lPEI nanoparticle assembly kinetics in FNC under turbulent mixing condition. Based on these findings and the nanoparticle assembly model mentioned above, the assembly kinetics under τ_{M}<τ_{A} condition could be analyzed to understand the concentration-dependent mechanism for determining pDNA copy number per nanoparticle (FIG. 4E). The rate of pDNA-lPEI binding (i.e., PEC unit formation) in Step 1 is much faster than the rate of PEC compaction and association in Step 2 (as a conclusion from FIG. 3A, FIG. 3B, FIG. 3C), such that τ_{Step 1}<<τ_{Step 2}, and τ_{A}≈τ_{Step 2}.

The characteristic assembly time τ_{A} is presumably influenced by intrinsic properties of the polyelectrolytes involved in nanoparticle assembly, such as plasmid length, lPEI structure and molecular weight, stoichiometric and steric nature of pDNA-lPEI binding, and the like. Upon generation of the turbulent flow structures (defined as t=0), mixing occurs primarily by lPEI molecules diffusing into the pDNA solution regions, and lPEIs diffusion proceeds on a time course of τ_{M}. The fast lPEI binding onto pDNAs happen with an N/P ratio of approximately 2.7 as lPEIs diffuse. When t≥τ_{M}, mixing completes and results in almost all pDNAs bound with the same amount of lPEIs, forming uniform PEC units that are about to proceed to Step 2 as the building blocks for nanoparticle assembly. The assembly occurs under the 0 condition (FIG. 3D, FIG. 4B, and Eq. 2), where PEC chain-chain interaction cancels the PEC-solvent interaction. There is no additional barrier for multi-PEC chain folding and association as opposed to single PEC chain folding. As a result, the compaction of PEC units ends with the same condensation degree regardless of the number of pDNA involved in the assembly of a single nanoparticle (i.e., regardless of the final N̅). An N̅>1 is possible when multiple PEC units are brought into contact by diffusion at a sufficiently fast rate for multi-PEC chains to be compacted into a single nanoparticle. Therefore, the number of PEC units that involves in the assembly of a single nanoparticle is dictated primarily by PEC diffusion within the time course of τ_{A}. A higher input pDNA concentration results in a higher pDNA concentration in the pDNA flow regions in the turbulent flow structures, thus a lower average distance between pDNA molecules in the solution, such that more PEC units could be associated within a time scale similar to τ_{A} as a result of a shorter diffusion distance between PEC units. Therefore, it is possible to explicitly control the number of pDNA to be packaged into a single nanoparticle under the kinetically controlled mixing conditions defined in the FNC process (i.e., when τ_{M}<τ_{A}).

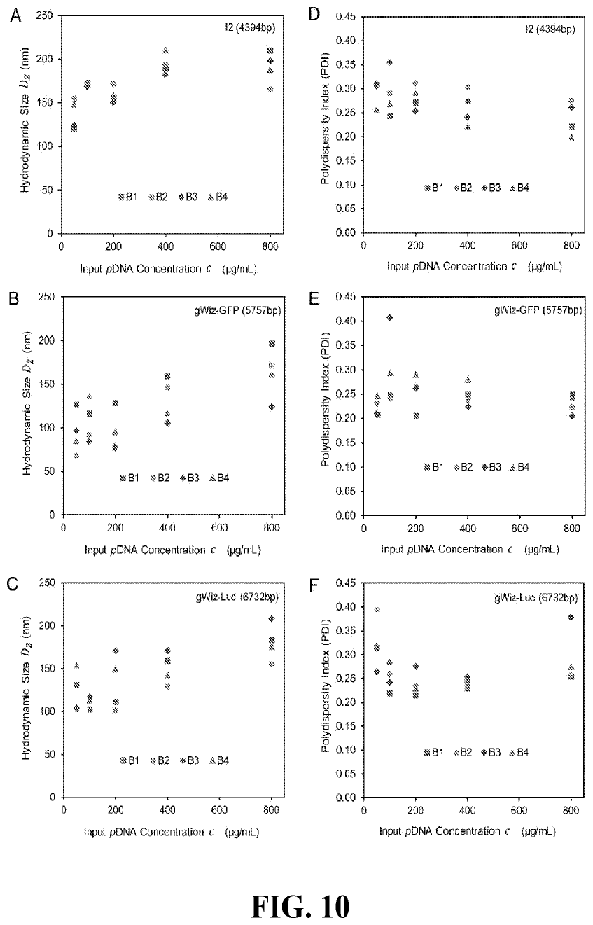

Based on the analysis above, if the input pDNA concentration is sufficiently low, that the average distance between any two pDNA molecules is too large for them to diffuse into each other over the time scale of τ_{A}, single plasmid containing nanoparticles can be produced. With the correlation of weight average molar mass and nanoparticle size (FIG. 4A), the extrapolated size limit when c→0 falls between 30 and 40 nm for the plasmids (4.4 kb to 6.7 kb) tested in this study (FIG. 2D), represents a typical size for pDNA/lPEI nanoparticles containing only one pDNA per nanoparticle. This small size and single pDNA payload were never achieved by pipetting, for which mixing kinetics were poorly controlled, as shown in FIG. 10 with the lowest input pDNA concentration of 25 μg/mL.

1.4.8. Transfection efficiency of pDNA/lPEI nanoparticles with different copy numbers of pDNA per nanoparticle. Using gWiz-Luc plasmid at different concentrations, nanoparticles were generated in the FNC device at a flow rate of 20 mL/min with N̅=1.7 (input c=100 μs/mL), 3.5 (input c=200 μg/mL), 6.1 (input c=400 μg/mL) and 21.8 (input c=800 μg/mL). With this series of nanoparticles, the effect of N̅ on the in vitro and in vivo transfection efficiency of these nanoparticles was examined. It is important to note that the sizes of these nanoparticles also are different, even though surface charges (zeta potentials) and compositions (bound and free lPEI fractions) are the same (Table 3).

TABLE 3
Summary of the characteristics of the nanoparticles with different average pDNA
copy number per nanoparticle (N̅) tested in vitro and in vivo

| | | | Zeta- | Bound PEI | Free PEI per |
|---|---|---|---|---|---|
| FNC input pDNA | | Hydrodynamic | potential* | Percentage | μg pDNA |
| Concentration | N̅ | Size* (nm) | (mV) | (%) | dose (μM) |
| 100 | 1.7 | 49.4 ± 0.5 | +42.3 ± 1.5 | 73.1 ± 0.7 | 3.24 ± 0.08 |
| 200 | 3.5 | 63.4 ± 0.5 | +41.1 ± 1.8 | 69.1 ± 1.2 | 3.64 ± 0.12 |
| 400 | 6.1 | 88.8 ± 0.6 | +41.6 ± 1.8 | 69.7 ± 2.6 | 3.63 ± 0.32 |
| 800 | 21.8 | 132.1 ± 0.6 | +39.7 ± 0.7 | 67.2 ± 2.1 | 3.94 ± 0.26 |

*Data are shown in a form of average ± standard deviation with n = 3 measurements.

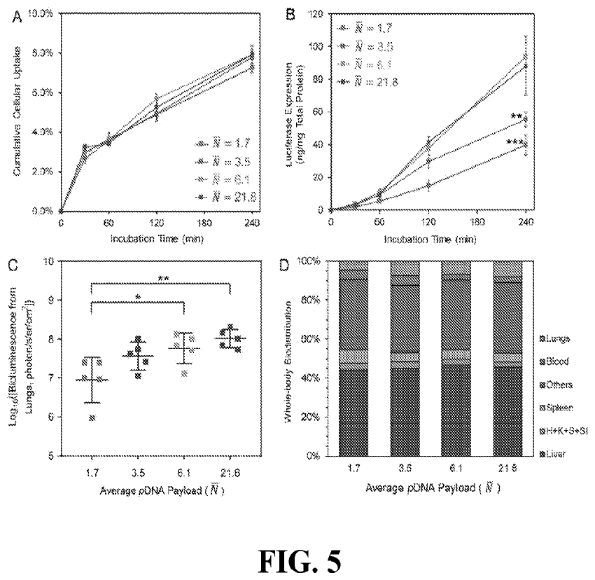

Previous reports demonstrate dependence of cellular uptake on nanoparticle size due to differences in surface contact, avidity and trafficking kinetics. Hickey et al., 2015. 3H-labelled pDNA were used to assemble nanoparticles (see Section 1.6) with different N̅ to assess their cellular uptake over a 4-h incubation period with PC3 prostate cancer cells. The data showed no difference among these nanoparticle groups (FIG. 5A). As the total pDNA dose was fixed (0.6 μg per 1×10⁴ cells with 5×10⁴ per well in a 24-well plate) for the transfection test, the measured total fraction of nanoparticle uptake (out of the total dosed nanoparticles) is a function of the total number of nanoparticles available per cell and uptake rate. Presumably, a formulation with higher N̅ has fewer nanoparticles in number and thus higher cellular uptake rate. With the similar uptake pDNA amount for each time point considered, N̅=6.1 and 21.8 may have higher efficiencies in intracellular delivery process, such as endosomal escape, pDNA dissociation, and nuclear transport. In previous literature reports, pDNA/lPEI nanoparticles typically give transfection and transgene activity in the lung following intravenous (i.v.) injection. Boeckle et al., 2004. In vitro transfection efficiency experiments in the PC3 cancer cell line showed that N̅=6.1 and 21.8 had a similar transfection efficiency levels that were much higher than those of either N̅=1.7 or 3.5 (FIG. 5B).

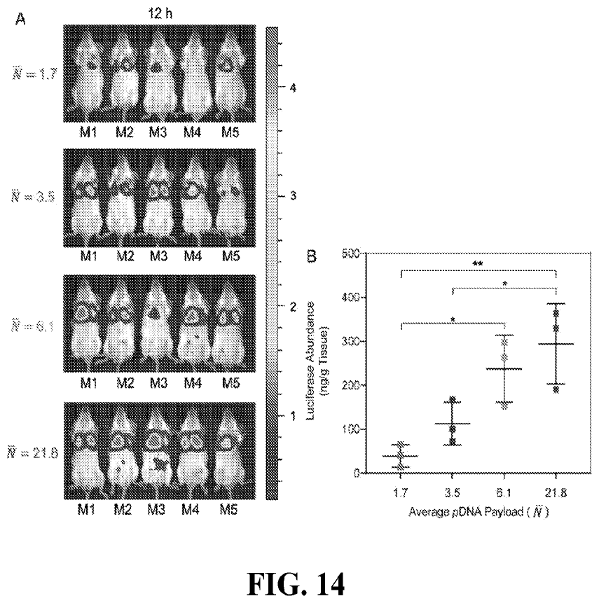

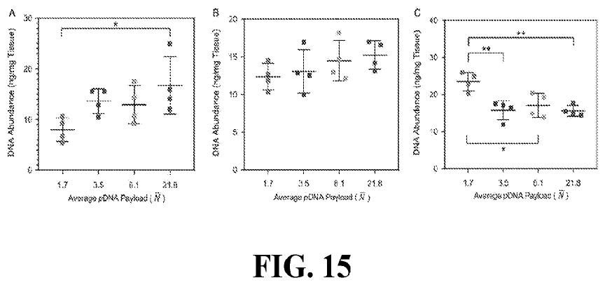

Consistent with the in vitro findings, N̅=1.7 showed an appreciably lower transfection efficiency than other formulations, and there was a perceived trend for nanoparticles with N̅=3.5, 6.1, and 21.8 that a higher N̅ gave better transfection efficiency (FIG. 5C and FIG. 14). A biodistribution study was conducted in Balb/c mice by i.v. injection of 3H-labeled nanoparticles at a dose of 30 μg pDNA/mouse. Mice were sacrificed at 1 h following dosing of the nanoparticles, and major organs and blood samples collected and weighted. The biological samples were solubilized, and the solutions were subjected to liquid scintillation assessments to quantify 3H-labeled pDNA in the samples. The results revealed a rapid distribution (>95%) of the nanoparticles into organs and tissues within 1 h for all formulations. The distribution patterns of these nanoparticles were similar except that those with an N̅ of 1.7 resulted in fewer nanoparticles deposited in the lung (FIG. 5D and FIG. 15A); and the clearance via the spleen was more significant (FIG. 15C). For all groups, even though 42-45% of the total dose ended up in the liver, comparing with 5-8% of the dose to the lung (FIG. 15B), there was no detectable level of transgene expression in the liver. This was probably due to the rapid clearance and degradation of nanoparticles by the Kupffer cells in the liver. Tsoi et al., 2016.

The smaller difference in in vivo delivery efficiency among these nanoparticles was likely due to the fact that pDNA/lPEI nanoparticles interact with serum component strongly and aggregate rapidly following i.v. injection, leading to entrapment in the lung microvasculatures and substantial uptake by endocytic cells in the liver and lung, Ogris et al., 1999, which would mask the differences of the payload capacity as a result of controlling N̅. Identifying nanoparticles (e.g., PEGylated nanoparticles) with lower tendency of opsonization and aggregation, and understanding the mechanism of serum coating will help to better reveal the detailed effect of nanoparticles with defined composition and size on transfection efficiency in vivo.

1.4.9. Effect of pDNA payload and PEI composition (i.e., bound vs. free PEI concentration) of pDNA/lPEI nanoparticles prepared under kinetically controlled conditions on their in vitro and in vivo transfection efficiency. The above pilot study revealed that nanoparticles with N̅ of 6 or higher showed better transfection efficiency in vitro and in vivo than nanoparticles with lower plasmid payload. Nanoparticles prepared under a turbulent mixing condition (Q=20 mL/min, τ_{M}=15 ms<τ_{A}) were then examined at different N/P ratios and compared them with those prepared at Q=5 mL/min, τ_{M}=790 ms>>τ_{A}). Two series of nanoparticles were prepared with gWiz-Luc plasmid, with detailed characteristics shown in Table 4.

TABLE 4
pDNA/lPE1 nanoparticles prepared with different
mixing conditions and N/P ratios

| Nanoparticle | | Q | Input | | |
|---|---|---|---|---|---|
| Code | Plasmid | (mL/min) | N/P | D_{z} (nm) | N̅ |
| W1 | gWiz-Luc | 20 | 3 | 88.8 ± 0.6 | 9.1 |
| W2 | | | 4 | 81.2 ± 0.2 | 6.1 |
| W3 | | | 5 | 79.3 ± 1.0 | 5.0 |
| W4 | | | 6 | 77.5 ± 0.7 | 4.4 |
| W5 | | 5 | 3 | 153.0 ± 3.7 | 40.7* |
| W6 | | | 4 | 158.9 ± 1.0 | 45.6* |
| W7 | | | 5 | 148.1 ± 0.9 | 37.0* |
| W8 | | | 6 | 155.5 ± 0.8 | 42.8* |
| W9 | | Pipetting | 6 | 171.2 ± 1.2 | 57.0* |
| P1 | PEG-fLuc | 20 | 3 | 81.4 ± 0.2 | 8.1* |
| P2 | | | 4 | 86.0 ± 0.9 | 9.5* |
| P3 | | | 5 | 82.7 ± 0.6 | 8.5* |
| P4 | | | 6 | 81.0 ± 0.1 | 8.0* |
| P5 | | 5 | 3 | 146.9 ± 1.9 | 45.7* |
| P6 | | | 4 | 151.1 ± 0.6 | 49.7* |
| P7 | | | 5 | 145.6 ± 2.9 | 44.5* |
| P8 | | | 6 | 134.1 ± 2.4 | 34.9* |

*Calculated based on Eqs. 2 and 7.

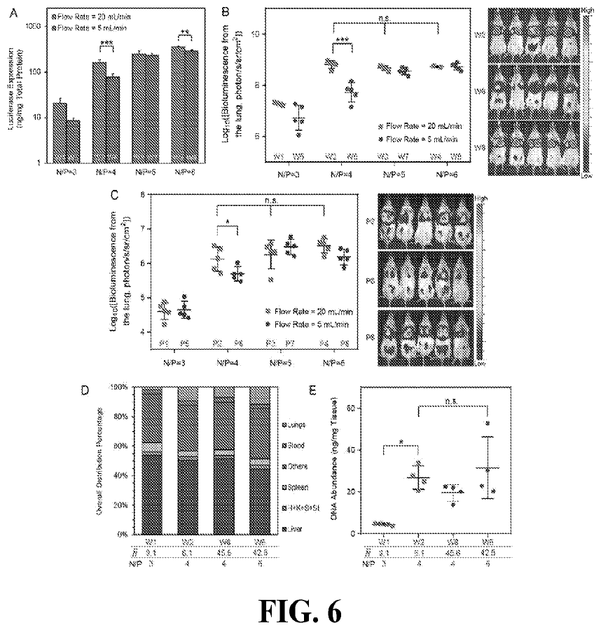

First, all nanoparticles prepared with gWiz-Luc plasmid were tested in the PC3 cancer cell line (FIG. 6A). For both sets of nanoparticles (W1-4 and W5-8), higher N/P ratio (i.e., higher free PEI fraction as a result) yielded higher transfection efficiency, which was in agreement with previous literature reports. Boeckle et al., 2004; Klauber et al., 2016.

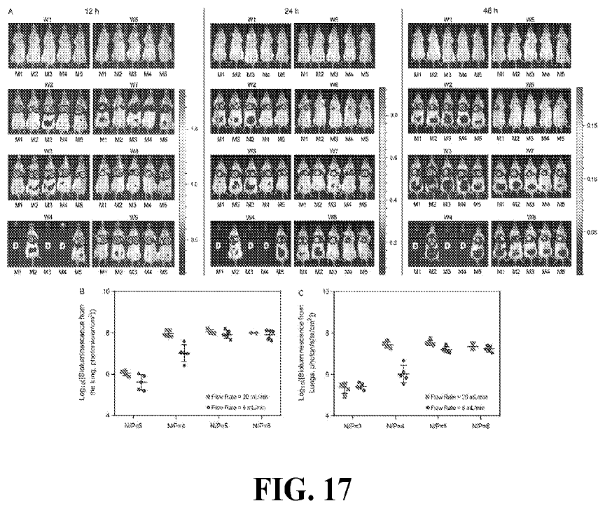

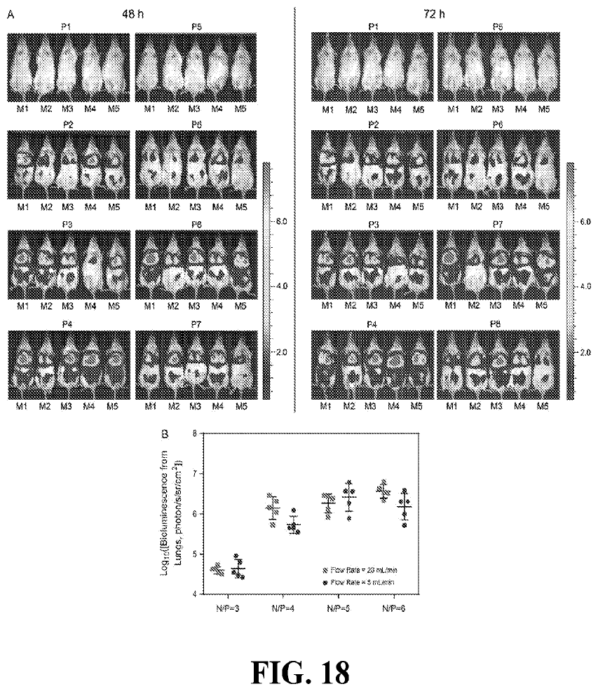

Nanoparticles with a lower payload showed better performances over those nanoparticles with higher payload, particularly for N/P=4 and 6. The same sets of nanoparticles were then administered to Balb/c mice and their transfection efficiencies in the lung were monitored at 12-h, 24-h and 48-h post-injection time points. The results (FIG. 6B and FIG. 17) showed a similar pattern as the in vitro experiments. The transgene expression activity was low for nanoparticles prepared at an N/P ratio of 3 for both low-payload and high-payload nanoparticles. With an N/P=4, nanoparticles with low payload (N̅ of =6.1) were more effective than nanoparticles with higher payload (N̅ of =45.6). The transgene activities for the N/P=4, N̅ of =6.1 formulation, and the nanoparticles prepared at N/P=5 and 6 showed similar luciferase expression levels regardless of the payload level N̅.

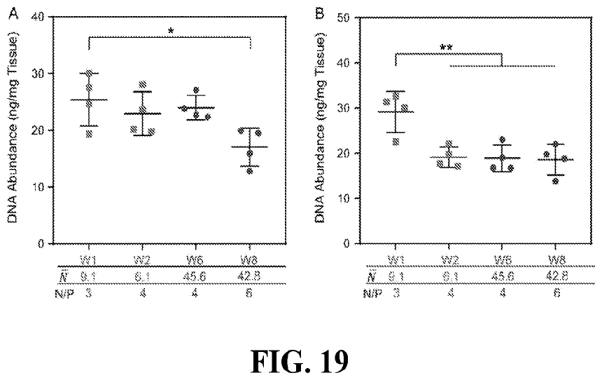

The biodistribution of four selected nanoparticle formulations from this series also was characterized, with the biggest differences in their transfection efficiencies: nanoparticles with lower payload and lower N/P ratios (Table 4, W1: N/P=3 and N̅=9.1, W2: N/P=4 and N̅=6.1), and nanoparticles with higher payload and higher N/P ratios (Table 4, W6: N/P=4 and N̅=45.6, W8: N/P=6 and N̅=42.8). The 3H-labeled nanoparticles were i.v. injected at the same dose as used for FIG. 6B and FIG. 6C in Balb/c mice. The majority (>95%) of the injected nanoparticle dose was distributed to tissues and organs within 1 h (FIG. 6E). W1 nanoparticles had the lowest fraction of nanoparticles distributed into the lung (1.4%) (FIG. 6D, FIG. 6E) with the highest levels of distribution to the liver (54.0%, though with no statistical significance) and spleen (6.0%) compared with other nanoparticle formulations (FIG. 19), correlating with the lowest transfection efficiency in the lung. W2 showed similar levels of distribution as W8 to the lung (FIG. 6D, FIG. 6E), which correlated with the similar transgene expression levels between these two formulations (FIG. 6C). W6, even though yielded a similar biodistribution profile as W2, gave a significantly lower transfection efficiency than W2. This may be attributed to less efficient intracellular delivery efficiency as shown in the in vitro study (FIG. 6A), and also a 7.5-fold fewer number of nanoparticles delivered to the lung. For lower payload nanoparticle formulations, the greater number of nanoparticles may facilitate more transfection events in a higher number of cells. On the other hand, the smaller size of these nanoparticles may influence nanoparticle trafficking in the tissue and the access to tumor tissues. Comparing W6 and W8 nanoparticles, even though their payloads (N̅=45.6 and 42.8) and sizes (Dg=158.9 and 155.5 nm) were similar, the higher level of free lPEI (FIG. 3B, 1.75 mM vs. 0.74 mM) led to a lower level of clearance by the liver (FIG. 6D and FIG. 19A). Together with a higher intracellular delivery efficiency (FIG. 6A), this factor may be responsible for a relatively higher transgene expression activity in the lung.

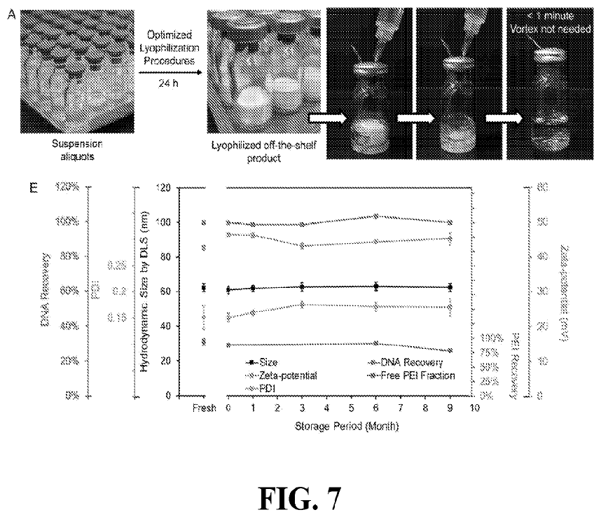

1.4.10. Scale-up production of an off-the-shelf, lyophilized pDNA/lPEI nanoparticles. Successful clinical translation of non-viral DNA delivery gene therapy depends on a high delivery and transfection efficiency, good biocompatibility, a scalable production processes, prolonged storage stability, and high performance consistency (i.e., low batch-to-batch variability). Current methods for systemic delivery of pDNA through PEC nanoparticles rely on on-site mixing of the therapeutic pDNA and in vivo-jetPEI® solutions in the clinic right before administration (i.e., similar to manual pipetting, as W9 in Table 4). As expected, the reproducibility of nanoparticle formation and performance consistency would be difficult to define (FIG. 9). Nanoparticle preparation by the FNC process reported here offers a continuous and highly scalable and reproducible method.15-17 With a single bench-model device, 0.5 grams of pDNA could be packaged into pDNA/lPEI nanoparticles within one hour, which is equivalent to 12,500 doses of 40 μg pDNA/mouse. The resulting nanoparticle suspensions can be subjected to an optimized lyophilization protocol to turn them into a powder form (FIG. 7A) that includes 9.5% w/w trehalose as a cryoprotectant agent. The lyophilized pDNA/lPEI nanoparticles were stable for at least 9 months when stored at −20° C. Upon reconstitution at each time point, the size, PDI, zeta-potential, PEI recovery, and DNA recovery were all consistent with the freshly prepared sample (FIG. 7B). The reconstitution process is simply by adding water, without the need of vortexing, generating a clear suspension without any aggregation after sitting for less than 1 minute at room temperature (FIG. 7A). The reconstituted pDNA/lPEI nanoparticles maintained the stability for at least 4 days.

1.5 Summary

The presently disclosed subject matter combines a simulation method and experimental approach to provide a detailed understanding of the kinetics of the mixing in the CU microchamber used for FNC assembly of pDNA/WEI nanoparticles that allows the flow rates of the input pDNA and lPEI solutions to be correlated with the characteristic mixing time. By controlling the mixing kinetics that enables the turbulent mixing of the solutions in the microchamber, an explicit control over the nanoparticle size, surface charge and compositions with high uniformity and scalability was demonstrated. From the static light scattering measurements and composition analysis of the FNC assembled pDNA/lPEI nanoparticles, a “universal” correlation between the nanoparticle hydrodynamic size and pDNA payload per nanoparticle was realized, suggesting that pDNA neutralization and compaction were achieved at the same degree for nanoparticles assembled with different pDNA payloads under different conditions. These findings not only allowed for finer composition control of pDNA/lPEI nanoparticles beyond control over nanoparticle size, but also provided experimental evidence to probe the kinetic process of pDNA/lPEI nanoparticle assembly. The charge neutralization between pDNA and lPEI molecules in forming PEC units was confirmed not to be a rate-limiting step, and the characteristic assembly time was found to be primarily determined by chain folding and compaction of the PEC units. The number of pDNA packaged into each nanoparticle is primarily determined by the diffusion distance of PEC units (i.e., the local pDNA concentration). By controlling the input pDNA concentration from 50 μg/mL to 800 μg/mL under kinetically controlled conditions by FNC, an average of about 1.7 pDNA to about 21.8 pDNA can be assembled correlating to the average hydrodynamic size of 35 to 130 nm. These well-defined nanoparticles enabled the investigation of the effect of pDNA payload and formulation composition on transfection efficiency of these nanoparticles. In the cancer cell models tested in this study, a medium payload of plasmid DNA in the nanoparticles was found to be optimal for the highest delivery efficiency in vivo and correlated well with their in vitro transfection activities. The in vivo transgene expression in both healthy and tumor bearing mouse models showed that a medium pDNA payload in pDNA/lPEI nanoparticles favors the transgene expression in the lung. These nanoparticles can be produced in a scalable manner as an off-the-shelf, lyophilized formulation that remains its stability for at least 9 months at −20° C. during storage. Moreover, this nano-formulation is easy to reconstitute and administer. Since this method does not specifically dependent on the carrier structure and plasmid length and type, it is generally applicable to many other potential polycation carriers. Thus, this FNC production process offers distinct technical advantages towards the clinical translation of non-viral nanoparticle vehicles for gene delivery.

1.6 Experimental

1.6.1. Preparations of pDNA/in vivo-jetPEI® polyelectrolyte complex (PEC) nanoparticles. All CIJ devices were manufactured by Johns Hopkins Whiting School of Engineering machine shop based on typical CIJ designs. Johnson and Prud'homme, 2003. In vivo-jetPEI® was used as received and diluted by ultrapure water to desired concentrations corresponding to different input N/P ratios from 3 to 6. The pH of the solutions (regardless of the concentration) was adjusted to 3.50 by NaOH or HCl to keep a consistent charge density on PEI molecules across all experiments. The pDNA was delivered in pure water by the manufacturer (Table 1) and diluted by ultrapure water to a concentration range from 50 to 800 μg/mL, when investigating the effect of input pDNA concentration on nanoparticles with input N/P ratio of 4. PEC nanoparticles were formulated by injection of the two working solutions into CIJ chamber with preset flow rates by a high-pressure syringe pump. The PEC nanoparticles are immediately stable upon exiting CIJ without any need of additional incubation time and were subjected to downstream characterizations and applications directly. When an isotonic condition is required, the pDNA and PEI working solutions were prepared in 9.5% (w/w) trehalose instead of water. All formulations were stable for at least one month in room temperature. For preparations of PEC nanoparticles (W9, Table 4) through pipetting, the procedure B3 was used as shown in Table 2.

1.6.2. Uncomplexed PEI assessments. The method was adopted from a previous report (Bertschinger et al., 2004). A 500-μL aliquot of diluted nanoparticle suspension was added into a Vivaspin 500 centrifugal concentrator (PES, MWCO of 100,000, Sartorius, n=4 concentrators for each sample). The concentrators were then centrifuged for 1 min to get flow-through solutions containing no nanoparticles but only uncomplexed PEI molecules. An aliquot of 60 μL of the flow-through solution was added into one well of a 96-well plate=3 wells for each filtered solution). Protein Red Advanced Protein Assay (PRAPA, Cytoskeleton US) solution (200 μL) was added into the well and the mixture was incubated at room temperature for 10 min.

1.6.3. Characterizations of the PEC nanoparticles. Dynamic light scattering (DLS) for size measurement and phase analysis light scattering (PALS) for zeta-potential measurement were conducted using a Malvern ZEN3690 Zetasizer at 25° C. As the most reliable outcome from the Zetasizer, the z-average hydrodynamic diameter was obtained and used as the size of the PEC nanoparticles for all the analysis in this study. As the polydispersity (PDI) given by the DLS machine (which follows procedures in ISO 13321) is dependent on nanoparticle size, we used a size distribution width to evaluate the uniformity of the nanoparticles in FIG. 2A with different sizes. This size distribution width is given directly by the DLS machine as a standardized term of size standard deviation, that for a single size peak:

$$Size distribution width = DLS size standard deviation = {( \frac{PDI}{{D}_{Z}^{2}} )}^{\frac{1}{2}} \qquad ( 4 )$$

Zeta-potential measurements were conducted in a low-salt buffer (5 mM NaCl) to give a conductivity of 0.6 mS/cm of the suspension for reliable assessments.

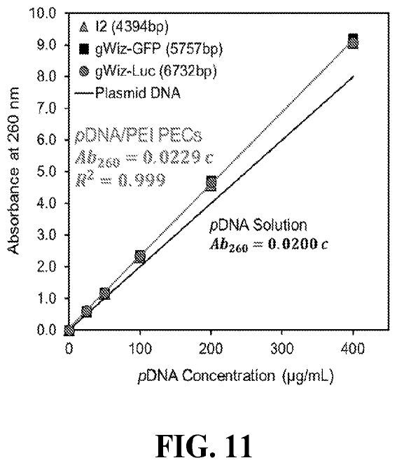

1.6.4. Static light scattering (SLS) was done on a Wyatt DAWN HELEOS 18-angel laser light scattering photometer, equipped with a laser source with the wavelength of 658 nm and a fused silica flow cell as the optical compartment. The machine was properly calibrated according to manual, with all the laser detectors normalized against an isotropic scatter (3 nm dextran, MW 9000-11000, Sigma US). PEC suspensions diluted to appropriate concentrations were introduced into the flow cell through a filter with size cut-off of either 450 nm or 1 μm. Each sample was run at a flow rate of 200 μL/min for 5 min to establish stable signals from the detectors. Data collection was followed for 5 min to give time-averaged intensities of each detector. The PEC nanoparticles coming out of the machine were collected and subjected to DLS and DNA recovery assessments (by NanoDrop, FIG. 11) to ensure unaffected nanoparticle characteristics and concentrations during the process. Data processing (generation of a Zimm or Debye plot) was conducted by the Wyatt ASTRA 6.1 software. n=3 independent runs were conducted to each sample, and the molar mass results presented herein were average values. The PEC nanoparticles can be treated as copolymers consisting of two components under SLS36 to give the weight average molar mass, as directed by light scattering theories. Hiemenz and Lodge, 2007.

To determine the refractive index increment (dn/dc) of the PEC nanoparticles, we followed the additive rule described previously (Dai and Wu, 2012):

$${( \frac{dn}{dc} )}_{Nanoparticle} = {{w}_{pDNA} ( \frac{dn}{dc} )}_{pDNA} + {{w}_{PEI} ( \frac{dn}{dc} )}_{PEI} \qquad ( 5 )$$

where w_{pDNA} and w_{PEI} are the weight fraction of pDNA and PEI complexed in PEC nanoparticles, respectively. The do/dc values are available by plugging in input pDNA concentrations and bound PEI fraction from results of free PEI assessments. Based on the proposed models for nanoparticle assembly by FNC, for each one of pDNA molecule, all the associated bound PEIs have a molar mass of:

$${M}_{Associated lPEI} = γ \times \frac{{c}_{m} ( PEI )}{{c}_{m} ( pDNA )} \times {M}_{pDNA} \qquad ( 6 )$$

where γ is bound PEI fraction given by free PEI assessments; cₘ(PEI) and cₘ(pDNA) are input mass concentrations of PEI and pDNA for formulation, respectively; and M_{DNA} is the molecular weight of the pDNA used. Then the (weight) average pDNA copy number per nanoparticle can be calculated by:

$$\overline{N} = \frac{{\overline{M}}_{Nanoparticle}}{{M}_{Associated lPEI} + {M}_{pDNA}} \qquad ( 7 )$$

where M̅_{Nanoparticle} is the weight average molar mass of the nanoparticles given by SLS.

1.6.5. Transmission electron microscopy (TEM). Carbon-coated copper grids (Electron Microscopy Services, US) were subjected to plasma treatment (N₂ glow discharge) for 30 sec before sample loading to render the film hydrophilic. The PEC nanoparticle suspensions were incubated on the grid for 20 min, then dried by filter papers. An aliquot of 10 μL of 2% (w/v) uranyl acetate solution was dropped onto the grid, incubated for 1 min and then dried by filter papers. The grids were allowed to be dried under hood for 24 h before imaging. As the dye solution has a low pH which renders uranyl groups positively charged to react strongly with pDNA molecules, a negative staining pattern (nanoparticles sitting on layer of dye with peripherals clearly marked) was searched and imaged. Imaging was performed on FEI Tecnai 12 Twin Transmission Electron Microscope operating at 100 kV. All images were taken by a Megaview III wide angle camera.

1.6.6. In vitro transfection activity. PC3 cancer cells were seeded in 24-well plates with a density of 5×10⁴ cells/well to form a mono-layer cell culture. After 24-h culture, the medium in each well was aspirated. An aliquot of 50 μL PEC nanoparticle suspension containing 3 μg pDNA were added into 500 μL fresh medium, vortexed for 20 sec to mix, and the whole mixture was added into each well. The cells were incubated with the PEC nanoparticles for 1 to 4 h. Upon incubation, the mixture was aspirated, and the cells were washed by PBS twice, and placed in fresh medium. After another 24 h of incubation to allow the cells to express the luciferase. For harvest, 100 μL of reporter lysis buffer (Promega, US) was added to each well, and the whole plate was subjected to two freezing-thawing cycles. Standard luciferase quantitative assay (Promega, US) and protein quantitative assay (Pierce BCA reagents, Thermo Scientific, US) were conducted to give the transfection efficiency in terms of ng of luciferase per mg of total protein in the lysate. For all tests, n=4 wells for each group tested.

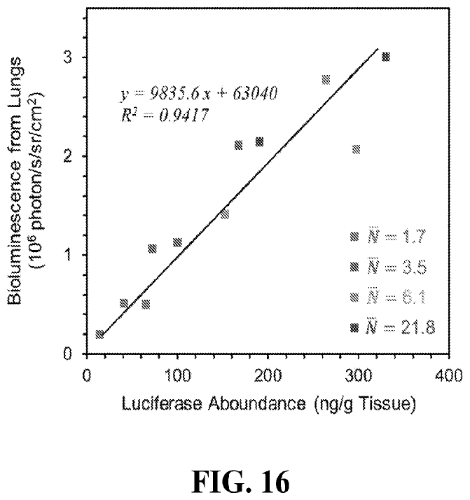

1.6.7. In vivo transfection efficiency. All in vivo experimental procedures were approved by the Johns Hopkins Institutional Animal Care and Use Committee (JHU ACUC). PEC nanoparticles were injected intravenously through mouse lateral tail vein with a dose of 30 or 40 μg pDNA per mouse at a concentration of 200 μg pDNA/mL. For groups with a lower input pDNA concentration in preparation, the nanoparticle suspensions were concentrated to 200 μg pDNA/mL by Amicon Ultra-2 centrifugal filter unit with a MWCO of 3,000 to concentrate both the PECs and the free PEI molecules with the same ratio. In vivo bioluminescence imaging was performed using the IVISR Spectrum (PerkinElmer, US) and the images were processed with Living Image Software (PerkinElmer, US). The region of interest (ROI) quantitative analysis results have good correlations with luciferase protein abundance in lungs that presents a linear relationship (FIG. 16), so tissue homogenization was not widely adopted for monitoring the kinetics of the transgene activities. Preliminary tests revealed that the transgene expression level (luciferase concentration in healthy lung tissues or tumor cells in the lung) peaks at around 12 and 48-h post injection for healthy Balb/c mouse model (Jackson Laboratory, US). IVIS assessment time points were set accordingly, with the mice anesthetized by isoflurane and imaged by IVIS system upon i.p. injection of 100 μL of 30 mg/mL D-luciferin (Gold Biotechnology, US) solution and 5-min diffusion period. For LL2 tumor model, inoculation was done through i.v. injection of 200 μL PBS solution containing 5×10⁵ cancer cells, 3 days prior to PEC dosage.

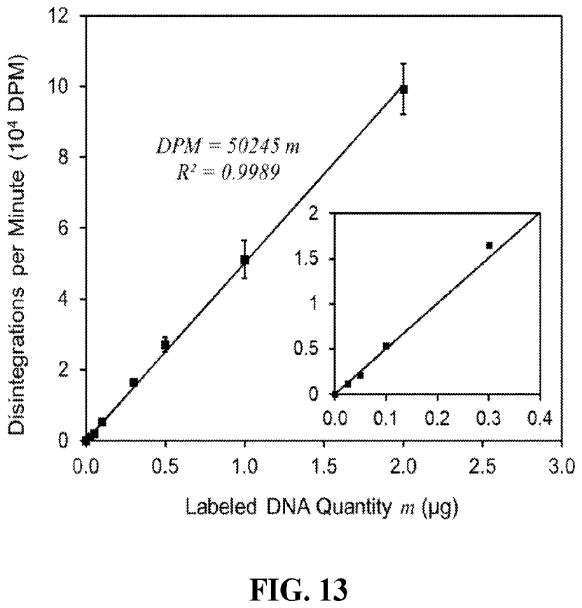

1.6.8. Preparation of 3H-labeled PEC nanoparticles. Cellular uptake and biodistribution studies. Tritium labelling of PEC nanoparticles was conducted by methylation of pDNAs by S-adenosyl-L[methyl-3H]methionine (3H-SAM) as the source prior to nanoparticle preparations. The processing results in minor structural change on pDNAs that does not affect the assembly process appreciably. The huge advantage of this labeling technique lies with the capability of assessing absolute quantity of labeled pDNAs in biological samples through counting disintegration events per minute (DPM) through scintillation fluid assay, thus ideal for cellular uptake and biodistribution research upon dosing the PEC nanoparticles. Within a working range, DPM is linearly proportional to labeled pDNA quantity in the assay (FIG. 13). To label pDNAs, water, 10×NEB buffer (New England Biolabs, US), 3H-SAM (PerkinElmer, US), pDNA (1 mg/mL) and M. Sssl enzyme (New England Biolabs, US) were added at a ratio of 12:2:2:1:1 (v/v) into a 50-mL tube.

The solution was mixed well, incubated for reaction at 37° C. for 2 h and quenched by heating to 65° C. for >30 min. The reaction mixture was diluted by EB buffer, with labeled pDNA purified using a QIAprep Spin Miniprep kit (Qiagen, US), and finally mixed with nonlabelled pDNA to give the working solution for PEC assembly. For cellular uptake experiments, the same dosing methods were adopted as in vitro transfection experiments. At each time point, the nanoparticle-containing medium was drained, and the cells were washed twice by fresh PBS and then harvested. For biodistribution studies, the same dosage and formulation concentration were adopted as in vivo transfection experiments. At 1-h post injection, the animals were sacrificed with tissues harvested and weighed. Sufficient SOLVABLE solubilization fluid (PerkinElmer, US) was added to tissues and incubate at 70° C. for 48 h. The tissue lysate was mixed well, and 100 μL of each sample (n=3 independent measurements) was added into 4 mL of Ultima Gold scintillation cocktail fluid (PerkinElmer, US) in 7-mL scintillation vials. DPM was assessed by a Tri-Carb 2200CA liquid scintillation analyzer (Packard Instrument Company, US) in a measurement time course of 5 min.

### Example 2

### Comparative Example—Composition of Flash Nanocomplexation FNC-Assembled Nanoparticles Compared to Nanoparticles Prepared by Bulk Mixing

As provided hereinabove, the composition of the presently disclosed DNA nanoparticles is unique in terms of the average number of DNA per particle, average particle size and size distribution, well-defined DNA and polymer content, and particle formulation in a lyophilized and shelf-stable form. Other reported DNA nanoparticle formulations do not have the exact same composition reports, so it is difficult to compare directly some of these benchmark parameters. There are specific physical property measurements, however, which could provide evidence on the distinct difference between the presently disclosed FNC-produced nanoparticles and those produced with bulk mixing methods, e.g., pipetting methods.

The presently disclosed FNC-assembled nanoparticles have distinct physical properties comparing with nanoparticles generated by pipetting method, a common bulk preparation method used at a laboratory scale. The pipetting method is provided herein as a comparative example of a bulk-mixing preparation. Note that the following results were prepared at a batch scale of 0.4 mL to 1.0 mL total volume. At a larger batch size, the resulting nanoparticles are much less well defined and more likely to generate aggregates.

Prepared with the same plasmid concentration at 200 μg/mL, pH of the lPEI solution at 3.5, trehalose concentration at 9.5% w/w, and NIP ratio at 4 or 6 (e.g., the minimum recommended level by the PEI manufacturer), FNC-assembled nanoparticles are more uniform with an average size close to about 80 nm, correlating to an average pDNA payload of 5 to 10 plasmids per nanoparticle (depending on the plasmid size) as compared to nanoparticles generated by bulk-mixing, which are less uniform, have a larger average size of 160 nm, and corresponding to an average pDNA payload of more than 40 plasmids per nanoparticle.

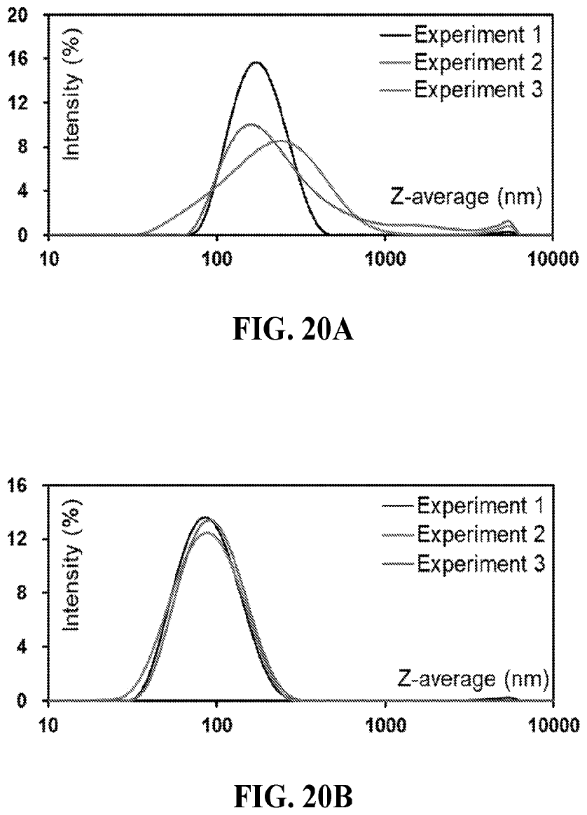

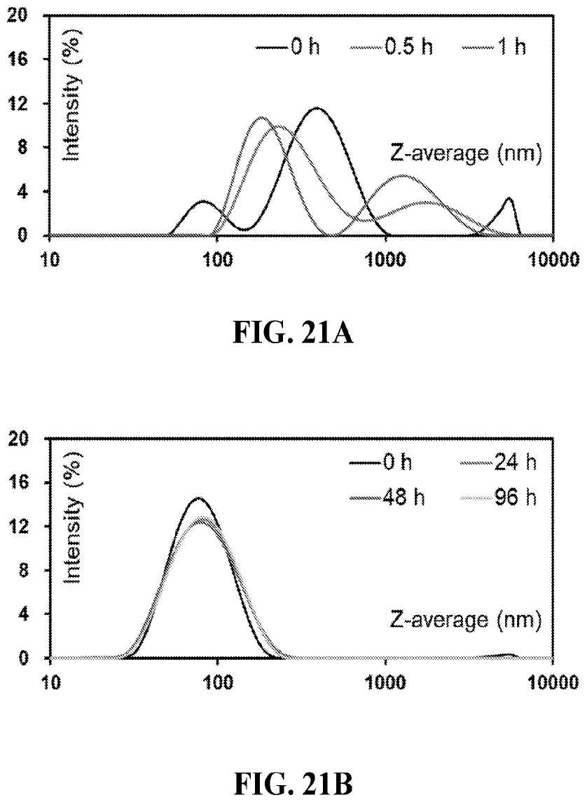

Moreover, the size and payload of nanoparticles generated by bulk-mixing methods known in the art (FIG. 20A) are much less reproducible and have higher degrees of variation compared to those prepared by the presently disclosed FNC method (FIG. 20B). These results indicate that the presently disclosed FNC methods provide much lower levels of batch-to-batch variation between preparations and between operators. Further, the quality of the nanoparticles depends on the exact practices of the pipetting, including which solution to pipette, pipetting speed, and how or if an additional mixing method, such as vortexing, is followed. Some pipetting practices result in aggregates that are not stable within hours of preparation (see FIG. 21A). In contrast, presently disclosed FNC-assembled nanoparticles are stable following production at room temperature for at least 96 hours (FIG. 21B).

More detailed comparisons between the presently disclosed FNC-nanoparticles and bulk-mixing generated nanoparticles are provided herein below in Table 6.

### Example 3

### Comparative Example—Transfection Efficiency and Toxicity of Flash Nanocomplexation FNC-Assembled Nanoparticles Compared to Nanoparticles Prepared by Bulk Mixing

The unique composition and properties of the presently disclosed FNC nanoparticles translate to a high transfection efficiency in comparison to nanoparticles prepared via bulk mixing under the same preparation conditions (N/P=4) (see Table 6). The presently disclosed FNC nanoparticles showed lower toxicity in vivo (Table 6). When dosing at 40 μg pDNA per mouse, the nanoparticles generated by bulk mixing at an N/P ratio of 6 resulted in severe toxicity compared to the FNC-assembled nanoparticles at an N/P ratio of 4, with 1/5 animal deaths shortly after injection, a higher level of alanine aminotransferase (ALT), and significant necrosis in the liver. Using FNC to produce nanoparticles at the same N/P ratio of 6 decreased the resultant necrotic area by nearly two folds. The FNC-assembled nanoparticles at an N/P ratio of 4 exhibited the lowest level of liver toxicity among all nanoparticle formulations, with the lowest increase of ALT serum level. Further, a low frequency of necrotic sites (<5%) as assessed by histological examinations was observed in the FNC nanoparticles.

TABLE 6
Comparison of FNC-Nanoparticles and Nanoparticles Prepared by Bulk Mixing.

| | | | | Pipetting | | Pipetting | |
|---|---|---|---|---|---|---|---|
| Method | | FNC-assembled | | (Bulk mixing) | | (Bulk mixing) | |
| Preparation | N/P ratio | 4 | | 4 | | 6 | |
| Condition | Plasmid | 200 | μg/mL | 200 | μg/mL | 200 | μg/mL |
| | concentration | | | | | | |
| | pH of lPEI in | 3.5 | | 3.5 | | 3.5 | |
| | preparation | | | | | | |
| | Flow Rate | 20 | mL/min | N/A | | N/A | |
| Composition | Size | 80 | nm | Varys | | Varys | |
| | | | | Always >160 nm | | Always >160 nm | |
| | pDNA per | 5-10 | | Always >40 | | Always >40 | |
| | nanoparticle | Depending on | | | | | |
| | | plasmid size | | | | | |
| | Free PEI | 0.7 | mM | 0.7 | mM | 1.8 | mM |
| | concentration | | | | | | |

| Physical | Uniformity | Uniform under TEM; | Heterogeneous under TEM; | |
|---|---|---|---|---|
| Properties | | PDI always <0.25 under | PDI usually >0.25 under DLS | |
| | | DLS | | |
| | Stability after | Stable for at least 4 days | May aggregate within hours with certain | |
| | preparation | in room temperature | pipetting practices under room temperature. | |
| Production | Reproducibility | Good | Poor | |
| throughput | between any two | Always <10 nm | Sometimes >60 nm | |
| | batches | | | |
| | Production | 480 mg pDNA per hour | 0.2 mg per batch (2 mL batch) | |
| | scalability | per device | 50 batches per hour (10 mg per hour) | |
| | | Continuous production | Batch mode | |
| Bioactivit | Transfection | High | Low | High |
| | efficiency in lungs* | | | |
| | Liver toxicity | Low | Mild to Moderate | Severe |
| | | Necrotic area <5% | Necrotic area ≥5% | Necrotic area >10% |
| | | for all mice | occasionally | for all mice |

*Tested at a dose of 40 μg DNA per mouse.

### Example 4

### Mammalian Cell Display

Mammalian display is a powerful method that is used for the selection of affinity reagents from combinatorial libraries of molecules expressed on the surface of cells. A library of plasmid DNA molecules can be used to express a library of different proteins that can be directed to the cell membrane using a leader sequence (Igκ, for example) and a membrane spanning region (PGFR, for example). The library of different binding domains, such as antibody variable regions, can then be exposed on the extracellular side of the membrane and are free to bind target ligands, for example cancer antigens. The target antigen can be labelled with biotin and detected with streptavidin-R-phycoerythrin, for example, so that cells expressing library members with suitable affinity for the ligand bind the ligand and the label and can be selected from the population of non-binding clones using flow cytometry. The DNA plasmids can then be isolated from the cells, transformed and grown in, for example, E. coli. The DNA plasmid clones can be transfected back into mammalian cells and the procedure repeated. Therefore, after several successive rounds of expression, binding, sorting and enrichment a small binding population from a large combinatorial population (containing binders and non-binders) can be isolated that has high affinity for the target ligand.

For the selection process to be efficient, a limited number of plasmids should be transfected per cell, otherwise both plasmids expressing non-binders and plasmids expressing binders would not be segregated sufficiently into different cells and would be enriched together in flow cytometry, because a heterogeneous population of antibody fragments, for example, would be expressed on each cell surface. Therefore, it is desirable to transfect one plasmid or few plasmids per cell so that one (or a few) antibody clones are expressed per cell and the clones expressing antibody fragments with highest affinity are selected efficiently at each cycle of selection by flow cytometry from non-binding clones and enrichment is achieved.

While methods for mammalian display are known in the art (see, e.g., Ho and Pastan, 2009), methods for achieving packaging of defined numbers of plasmids per nanoparticle are not. The Example provided hereinabove describe a method to package between about 1 plasmid to about 50 plasmids per nanoparticle and achieve high levels of transfection. For library purposes using between about 1 nanoparticle and about 10 nanoparticles (containing between about 1 plasmid to about 50 plasmids) per cell would be suitable for achieving transfection of a low number of plasmid clones per cell and therefore achieve efficient enrichment in mammalian display methods.

### REFERENCES

All publications, patent applications, patents, and other references mentioned in the specification are indicative of the level of those skilled in the art to which the presently disclosed subject matter pertains. All publications, patent applications, patents, and other references (e.g., websites, databases, etc.) mentioned in the specification are herein incorporated by reference in their entirety to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Barreleiro, P. C. A.; Lindman, B., The Kinetics of DNA—Cationic Vesicle Complex Formation. The Journal of Physical Chemistry B 2003, 107 (25), 6208-6213.
Baum, C.; O. Kustikova, U. Modlich, Z. Li, B. Fehse, Hum Gene Ther 2006, 17, 253.
Beh, C. W.; Pan, D.; Lee, J.; Jiang, X.; Liu, K. J.; Mao, H. Q.; Wang, T. H. Direct interrogation of DNA content distribution in nanoparticles by a novel microfluidics-based single-particle analysis. Nano Lett 2014, 14, 4729-35.
Berret, J.-F., Evidence of overcharging in the complexation between oppositely charged polymers and surfactants. The Journal of Chemical Physics 2005, 123 (16), 164703.
Bertrand, N.; J. Wu, X. Xu, N. Kamaly, O. C. Farokhzad, Advanced Drug Delivery Reviews 2014, 66, 2.
Bertschinger, M.; Chaboche, S.; Jordan, M.; Wurm, F. M., A spectrophotometric assay for the quantification of polyethylenimine in DNA nanoparticles. Anal Biochem 2004, 334 (1), 196-198.
Bessis, N., GarciaCozar, F. J., Boissier, M. C., Gene Ther 2004, 11 Suppl 1, S10.
Bhang, H.-e. C.; Gabrielson, K. L.; Laterra, J.; Fisher, P. B.; Pomper, M. G., Tumor-specific imaging through progression elevated gene-3 promoter-driven gene expression. Nature Medicine 2010, 17, 123.
Blanco, E.; Shen, H.; Ferrari, M., Principles of nanoparticle design for overcoming biological barriers to drug delivery. Nature Biotechnology 2015, 33, 941.
Boeckle, S.; von Gersdorff, K.; van der Piepen, S.; Culmsee, C.; Wagner, E.; Ogris, M., Purification of polyethylenimine polyplexes highlights the role of free polycations in gene transfer. The Journal of Gene Medicine 2004, 6 (10), 1102-1111.
Bonnet, M.-E.; P. Erbacher, A.-L. Bolcato-Bellemin, Pharmaceutical Research 2008, 25, 2972.
Buscail, L.; B. Bournet, F. Vernejoul, G. Cambois, H. Lulka, N. Hanoun, M. Dufresne, A. Meulle, A. Vignolle-Vidoni, L. Ligat, N. Saint-Laurent, F. Pont, S. Dejean, M. Gayral, F. Martins, J. Torrisani, O. Barbey, F. Gross, R. Guimbaud, P. Otal, F. Lopez, G. Tiraby, P. Cordelier, Mol Ther 2015, 23, 779.
Chauhan, V. P., Jain, R. K., Nature Materials 2013, 12, 958.
Chollet, P.; Favrot, M. C.; Hurbin, A.; Coll, J.-L., Side-effects of a systemic injection of linear polyethylenimine—DNA complexes. The Journal of Gene Medicine 2002, 4 (1), 84-91.
Curtis, K. A.; Miller, D.; Millard, P.; Basu, S.; Horkay, F.; Chandran, P. L., Unusual Salt and pH Induced Changes in Polyethylenimine Solutions. PLOS ONE 2016, 11(9), e0158147.
D'Addio, S. M.; Prud'homme, R. K. Controlling drug nanoparticle formation by rapid precipitation. Advanced Drug Delivery Reviews 2011, 63, 417-426.
D'Addio, S. M.; Saad, W.; Ansell, S. M.; Squiers, J. 1.; Adamson, D. H.; Herrera-Alonso, M.; Wohl, A. R.; Hoye, T. R.; Macosko, C. W.; Mayer, L. D.; Vauthier, C.; Prud'homme, R. K. Effects of block copolymer properties on nanocarrier protection from in vivo clearance. Journal of controlled release: official journal of the Controlled Release Society 2012, 162, 208-217.
D'Addio, S. M.; Baldassano, S.; Shi, L.; Cheung, L. L.; Adamson, D. H.; Bruzek, M.; Anthony, J. E.; Laskin, D. L.; Sinko, P. J.; Prud'homme, R. K. Optimization of cell receptor-specific targeting through multivalent surface decoration of polymeric nanocarriers. Journal of controlled release: official journal of the Controlled Release Society 2013, 168, 41-49.
Dai, Z.; Wu, C., How Does DNA Complex with Polyethylenimine with Different Chain Lengths and Topologies in Their Aqueous Solution Mixtures? Macromolecules 2012, 45 (10), 4346-4353.
Dubin, P.; Bock, J.; Davis, R.; Schulz, D. N.; Thies, C., Macromolecular complexes in chemistry and biology. Springer Science & Business Media: 2012.
Feng, Q.; Sun, J.; Jiang, X., Microfluidics-mediated assembly of functional nanoparticles for cancer related pharmaceutical applications. Nanoscale 2016, 8 (25), 12430-12443.

Gindy, M. E.; Ji, S. X.; Hoye, T. R.; Panagiotopoulos, A. Z.; Prud'homme, R. K. Preparation of Poly(ethylene glycol) Protected Nanoparticles with Variable Bioconjugate Ligand Density. Biomacromolecules 2008, 9, 2705-2711.
Ginn, S. L.; I. E. Alexander, M. L. Edelstein, M. R. Abedi, J. Wixon, J Gene Med 2013, 15, 65.
Han, J.; Zhu, Z. X.; Qian, H. T.; Wohl, A. R.; Beaman, C. 1.; Hoye, T. R.; Macosko, C. W. A simple confined impingement jets mixer for flash nanoprecipitation. J Pharm Sci-Us 2012, 101, 4018-4023.
He, Z.; Santos, J. L.; Tian, H.; Huang, H.; Hu, Y.; Liu, L.; Leong, K. W.; Chen, Y.; Mao, H.-Q., Scalable fabrication of size-controlled chitosan nanoparticles for oral delivery of insulin. Biomaterials 2017, 130, 28-41.
He, Z.; Hu, Y.; Nie, T.; Tang, H.; Zhu, J.; Chen, K.; Liu, L.; Leong, K. W.; Chen, Y.; Mao, H.-Q., Size controlled lipid nanoparticle production using turbulent mixing to enhance oral DNA delivery. Acta Biomaterialia 2018, 81, 195-207.
Hickey, J. W.; Santos, J. L.; Williford, J.-M.; Mao, H.-Q., Control of polymeric nanoparticle size to improve therapeutic delivery. Journal of Controlled Release 2015, 219, 536-547.
Hiemenz, P. C.; Lodge, T. P., Polymer chemistry. CRC press: 2007.
Ho, M.; Pastan, I. Mammalian Cell Display for Antibody Engineering. Methods Mol Biol. 2009; 525: 337-52.
Ho, Y. P.; Grigsby, C. L.; Zhao, F.; Leong, K. W. Tuning Physical Properties of Nanocomplexes through Microfluidics-Assisted Confinement. Nano Lett 2011, 11, 2178-2182.
Jere, D.; H. L. Jiang, R. Arote, Y. K. Kim, Y. J. Choi, M. H. Cho, T. Akaike, C. S. Cho, Expert Opinion on Drug Delivery 2009, 6, 827.
Johnson, B. K.; Prud'homme, R. K. Chemical processing and micromixing in confined impinging jets. Aiche J 2003, 49, 2264-2282.
Johnson, B. K.; Prud'homme, R. K. Flash NanoPrecipitation of organic actives and block copolymers using a confined impinging jets mixer. Aust J Chem 2003, 56, 1021-1024.
Johnson, B. K.; Prud'homme, R. K. Mechanism for rapid self-assembly of block copolymer nanoparticles. Physical review letters 2003, 91, 118302.
Juul, S.; Nielsen, C. J. F.; Labouriau, R.; Roy, A.; Tesauro, C.; Jensen, P. W.; Harmsen, C.; Kristoffersen, E. L.; Chiu, Y.-L.; Frohlich, R.; Fiorani, P.; Cox-Singh, J.; Tordrup, D.; Koch, J.; Bienvenu, A.-L.; Desideri, A.; Picot, S.; Petersen, E.; Leong, K. W.; Ho, Y.-P.; Stougaard, M.; Knudsen, B. R., Droplet Microfluidics Platform for Highly Sensitive and Quantitative Detection of Malaria-Causing Plasmodium Parasites Based on Enzyme Activity Measurement. ACS Nano 2012, 6 (12), 10676-10683.
Kamaly, N.; Xiao, Z.; Valencia, P. M.; Radovic-Moreno, A. F.; Farokhzad, O. C. Targeted polymeric therapeutic nanoparticles: design, development and clinical translation. Chemical Society reviews 2012, 41, 2971-3010.
Kasper, J. C.; Schaffert, D.; Ogris, M.; Wagner, E.; Friess, W., The establishment of an up-scaled micromixer method allows the standardized and reproducible preparation of well-defined plasmid/LPEI polyplexes. European Journal of Pharmaceutics and Biopharmaceutics 2011, 77 (1), 182-185.
Klauber, T. C. B.; Sondergaard, R. V.; Sawant, R. R.; Torchilin, V. P.; Andresen, T. L., Elucidating the role of free polycations in gene knockdown by siRNA polyplexes. Acta Biomaterialia 2016, 35, 248-259.
Kolishetti, N.; Dhar, S.; Valencia, P. M.; Lin, L. Q.; Kamik, R.; Lippard, S. J.; Langer, R.; Farokhzad, O. C. Engineering of self-assembled nanoparticle platform for precisely controlled combination drug therapy. Proceedings of the National Academy of Sciences of the United States of America 2010, 107, 17939-44.
Lai, E.; van Zanten, J. H., Monitoring DNA/Poly-l-Lysine Polyplex Formation with Time-Resolved Multiangle Laser Light Scattering. Biophysical Journal 2001, 80 (2), 864-873.
Lewis, D. R.; Petersen, L. K.; York, A. W.; Zablocki, K. R.; Joseph, L. B.; Kholodovych, V.; Prud'homme, R. K.; Uhrich, K. E.; Moghe, P. V. Sugar-based amphiphilic nanoparticles arrest atherosclerosis in vivo. P Natl Acad Sci USA 2015, 112, 2693-2698.
Lim, J. M., A. Swami, L. M. Gilson, S. Chopra, S. Choi, J. Wu, R. Langer, R. Karnik, O. C. Farokhzad, ACS Nano 2014, 8, 6056.
Liu, D.; Cito, S.; Zhang, Y.; Wang, C.-F.; Sikanen, T. M.; Santos, H. A., A Versatile and Robust Microfluidic Platform Toward High Throughput Synthesis of Homogeneous Nanoparticles with Tunable Properties. Advanced Materials 2015, 27 (14), 2298-2304.
Liu, D.; Zhang, H.; Cito, S.; Fan, J.; Makila, E.; Salonen, J.; Hirvonen, J.; Sikanen, T. M.; Weitz, D. A.; Santos, H. A., Core/Shell Nanocomposites Produced by Superfast Sequential Microfluidic Nanoprecipitation. Nano Letters 2017, 17 (2), 606-614.
Liu, Y.; Fox, R. O., CFD predictions for chemical processing in a confined impinging-jets reactor. AIChE Journal 2006, 52 (2), 731-744.
Liu, Y.; Cheng, C.; Liu, Y.; Prud'homme, R. K.; Fox, R. O., Mixing in a multi-inlet vortex mixer (MIVM) for flash nano-precipitation. Chemical Engineering Science 2008, 63 (11), 2829-2842.
Lu, M.; Ho, Y.-P.; Grigsby, C. L.; Nawaz, A. A.; Leong, K. W.; Huang, T. J., Three-Dimensional Hydrodynamic Focusing Method for Polyplex Synthesis. ACS Nano 2014, 8 (1), 332-339.
Lu, M.; Ozcelik, A.; Grigsby, C. L.; Zhao, Y.; Guo, F.; Leong, K. W.; Huang, T. J., Microfluidic hydrodynamic focusing for synthesis of nanomaterials. Nano Today 2016, 11 (6), 778-792.
Luo, H. Y.; Santos, J. L.; Herrera-Alonso, M. Toroidal structures from brush amphiphiles. Chem Commun 2014, 50, 536-538.
Mangraviti, A.; Tzeng, S. Y.; Kozielski, K. L.; Wang, Y.; Jin, Y.; Gullotti, D.; Pedone, M.; Buaron, N.; Liu, A.; Wilson, D. R.; Hansen, S. K.; Rodriguez, F. 1.; Gao, G. D.; DiMeco, F.; Brem, H.; Olivi, A.; Tyler, B.; Green, J. J. Polymeric nanoparticles for nonviral gene therapy extend brain tumor survival in vivo. ACS Nano 2015, 9, 1236-49.
Mastorakos, P.; da Silva, A. L.; Chisholm, J.; Song, E.; Choi, W. K.; Boyle, M. P.; Morales, M. M.; Hanes, J.; Suk, J. S. Highly compacted biodegradable DNA nanoparticles capable of overcoming the mucus barrier for inhaled lung gene therapy. Proceedings of the National Academy of Sciences of the United States of America 2015.
Minn, I.; Bar-Shir, A.; Yarlagadda, K.; Bulte, J. W. M.; Fisher, P. B.; Wang, H.; Gilad, A. A.; Pomper, M. G., Tumor-specific expression and detection of a CEST reporter gene. Magnetic Resonance in Medicine 2015, 74 (2), 544-549.
Mittal, R.; Dong, H.; Bozkurttas, M.; Najjar, F. M.; Vargas, A.; von Loebbecke, A., A versatile sharp interface immersed boundary method for incompressible flows with complex boundaries. Journal of Computational Physics 2008, 227 (10), 4825-4852.
Mura, S., J. Nicolas, P. Couvreur, Nature Materials 2013, 12, 991.
Murday, J. S.; Siegel, R. W.; Stein, J.; Wright, J. F. Translational nanomedicine: status assessment and opportunities. Nanomedicine: nanotechnology, biology, and medicine 2009, 5, 251-73.
Nikoubashman, A.; Lee, V. E.; Sosa, C.; Prud'homme, R. K.; Priestley, R. D.; Panagiotopoulos, A. Z., Directed Assembly of Soft Colloids through Rapid Solvent Exchange. ACS Nano 2016, 10 (1), 1425-1433.
Ogris, M.; Brunner, S.; Schuller, S.; Kircheis, R.; Wagner, E., PEGylated DNA/transferrin—PEI complexes: reduced interaction with blood components, extended circulation in blood and potential for systemic gene delivery. Gene Therapy 1999, 6, 595.
Osada, K.; Shiotani, T.; Tockary, T. A.; Kobayashi, D.; Oshima, H.; Ikeda, S.; Christie, R. J.; Itaka, K.; Kataoka, K., Enhanced gene expression promoted by the quantized folding of pDNA within polyplex micelles. Biomaterials 2012, 33 (1), 325-332.
Pack, D. W., Hoffman, A. S., Pun, S., Stayton, P. S., Nature Reviews Drug Discovery 2005, 4, 581.
Pagels, R. F.; Edelstein, J.; Tang, C.; Prud'homme, R. K., Controlling and Predicting Nanoparticle Formation by Block Copolymer Directed Rapid Precipitations. Nano Letters 2018, 18 (2), 1139-1144.
Patnaik, S., K. C. Gupta, Expert Opinion on Drug Delivery 2013, 10, 215.
Peer, D.; J. M. Karp, S. Hong, O. C. FaroKhzad, R. Margalit, R. Langer, Nature Nanotechnology 2007, 2, 751.
Romanowsky, M. B.; Abate, A. R.; Rotem, A.; Holtze, C.; Weitz, D. A. High throughput production of single core double emulsions in a parallelized microfluidic device. Lab on a chip 2012, 12, 802-7.
Saad, W. S.; Prud'homme, R. K., Principles of nanoparticle formation by flash nanoprecipitation. Nano Today 2016, 11 (2), 212-227.
Santhiya, D.; Dias, R. S.; Dutta, S.; Das, P. K.; Miguel, M. G.; Lindman, B.; Maiti, S., Kinetic Studies of Amino Acid-Based Surfactant Binding to DNA. The Journal of Physical Chemistry B 2012, 116 (20), 5831-5837.
Santos, J. L.; Herrera-Alonso, M. Kinetically Arrested Assemblies of Architecturally Distinct Block Copolymers. Macromolecules 2014, 47, 137-145.
Santos, J. L.; Ren, Y.; Vandermark, J.; Archang, M. M.; Williford, J.-M.; Liu, H.-W.; Lee, J.; Wang, T.-H.; Mao, H.-Q., Continuous Production of Discrete Plasmid DNA-Polycation Nanoparticles Using Flash Nanocomplexation. Small 2016, 12 (45), 6214-6222.
Seo, J. H.; Mittal, R., A sharp-interface immersed boundary method with improved mass conservation and reduced spurious pressure oscillations. Journal of Computational Physics 2011, 230 (19), 7347-7363.
Shen, H.; Hong, S. Y.; Prud'homme, R. K.; Liu, Y. Self-assembling process of flash nanoprecipitation in a multi-inlet vortex mixer to produce drug-loaded polymeric nanoparticles. J Nanopart Res 2011, 13, 4109-4120.
Shi, B.; Zheng, M.; Tao, W.; Chung, R.; Jin, D.; Ghaffari, D.; Farokhzad, O. C., Challenges in DNA Delivery and Recent Advances in Multifunctional Polymeric DNA Delivery Systems. Biomacromolecules 2017, 18 (8), 2231-2246.
Takeda, K. M.; Osada, K.; Tockary, T. A.; Dirisala, A.; Chen, Q.; Kataoka, K., Poly(ethylene glycol) Crowding as Critical Factor To Determine pDNA Packaging Scheme into Polyplex Micelles for Enhanced Gene Expression. Biomacromolecules 2017, 18 (1), 36-43.
Tennekes, H.; Lumley, J. L.; Lumley, J., A first course in turbulence. MIT press: 1972.
Tsoi, K. M.; MacParland, S. A.; Ma, X.-Z.; Spetzler, V. N.; Echeverri, J.; Ouyang, B.; Fadel, S. M.; Sykes, E. A.; Goldaracena, N.; Kaths, J. M.; Conneely, J. B.; Alman, B. A.; Selzner, M.; Ostrowski, M. A.; Adeyi, O. A.; Zilman, A.; McGilvray, I. D.; Chan, W. C. W., Mechanism of hard-nanomaterial clearance by the liver. Nature Materials 2016, 15, 1212.
Valencia, P. M.; Farokhzad, O. C.; Karnik, R.; Langer, R. Microfluidic technologies for accelerating the clinical translation of nanoparticles. Nat Nanotechnol 2012, 7, 623-9.
Wightman, L., Kircheis, R., Rossler, V., Carotta, S., Ruzicka, R., Kursa, M., Wagner, E., Journal of Gene Medicine 2001, 3, 362.
Williford, J.-M.; Santos, J. L.; Shyam, R.; Mao, H.-Q., Shape control in engineering of polymeric nanoparticles for therapeutic delivery. Biomaterials Science 2015, 3 (7), 894-907.
Yang, J.; Hendricks, W.; Liu, G. S.; McCaffery, J. M.; Kinzler, K. W.; Huso, D. L.; Vogelstein, B.; Zhou, S. B. A nanoparticle formulation that selectively transfects metastatic tumors in mice. P Natl Acad Sci USA 2013, 110, 14717-14722.
Yin, H.; R. L. Kanasty, A. A. Eltoukhy, A. J. Vegas, J. R. Dorkin, D. G. Anderson, Nat Rev Genet 2014, 15, 541.
Yue, Y.; Jin, F.; Deng, R.; Cai, J.; Chen, Y.; Lin, M. C. M.; Kung, H.-F.; Wu, C., Revisit complexation between DNA and polyethylenimine—Effect of uncomplexed chains free in the solution mixture on gene transfection. Journal of Controlled Release 2011, 155 (1), 67-76.
Zhang, C.; Pansare, V. J.; Prud'homme, R. K.; Priestley, R. D., Flash nanoprecipitation of polystyrene nanoparticles. Soft Matter 2012, 8 (1), 86-93.
Zhu, Z. X. Flash Nanoprecipitation: Prediction and Enhancement of Particle Stability via Drug Structure. Molecular pharmaceutics 2014, 11, 776-786.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for preparing uniform polyelectrolyte complex (PEC) nanoparticles, the method comprising homogeneously mixing one or more water-soluble polycationic polymers with one or more water-soluble polyanionic polymers under conditions having a characteristic assembly time (τ_{A}), over which assembly of the PEC nanoparticles occurs, greater than a characteristic mixing time (τ_{M}), over which the one or more water-soluble polycationic polymers and the one or more water-soluble polyanionic polymers are mixed homogenously.

2. The method of claim 1, wherein the method comprises a flash nanocomplexation (FNC) method.

3. The method of claim 2, wherein the method comprises: A
(a) flowing a first stream comprising one or more water-soluble polycationic polymers at a first variable flow rate into a confined chamber;
(b) flowing a second stream comprising one or more water-soluble polyanionic polymers at a second variable flow rate into the confined chamber, wherein the first stream and the second stream are on opposing sides when entering the confined chamber; and
(c) optionally flowing a third stream comprising one or more components selected from the group consisting of one or more water-soluble therapeutic agents, one or more miscible organic solvents, and/or one or more cryoprotectants at a third variable flow rate into the confined chamber; wherein each stream is equidistant from the other two streams when entering the confined chamber;
wherein the first variable flow rate, the second variable flow rate, and the third variable flow rate, if present, can be the same or different; and
(d) impinging the first stream, the second stream, and the third stream, if present, in the confined chamber until the Reynolds number is from about 1,000 to about 20,000, thereby causing the one or more water-soluble polycationic polymers and the one or more water-soluble polyanionic polymers to undergo a polyelectrolyte complexation process that continuously generates PEC nanoparticles, wherein the polyelectrolyte complexation process occurs under conditions having a characteristic assembly time (τ_{A}), over which assembly of the PEC nanoparticles occurs, which is greater than a characteristic mixing time (τ_{M}), over which components of the first stream, second stream, and third stream, if present, are mixed homogenously.

4. The method of claim 3, wherein the first variable flow rate, the second variable flow rate, and the third variable flow rate, if present, are each equal to or greater than about 10 milliliters/minute (mL/min).

5. The method of claim 3 or claim 4, wherein the first variable flow rate, the second variable flow rate, and the third variable flow rate, if present, are each between about 3 mL/min to about 30 mL/min.

6. The method of any of claims 3-5, wherein the characteristic mixing time is between about 1 ms to about 200 ms.

7. The method of any of claims 1-6, wherein the characteristic mixing time is about 15 ms.

8. The method of any of claims 3-7, wherein the Reynolds number has a range from about 2,000 to about 5,000.

9. The method of any of claims 3-8, wherein the pH value of the first stream and the pH value of the second stream each has a range from about 2.5 to about 8.4.

10. The method of any of claims 3-9, wherein the pH value of the first stream and the pH value of the second stream each is about 3.5.

11. The method of any of claims 1-10, wherein the one or more water-soluble polycationic polymers are selected from the group consisting of chitosan, PAMAM dendrimers, polyethylenimine (PEI), protamine, poly(arginine), poly(lysine), poly(beta-aminoesters), cationic peptides and derivatives thereof.

12. The method of any of claims 1-11, wherein the one or more water-soluble polyanionic polymers are selected from the group consisting of poly(aspartic acid), poly(glutamic acid), negatively charged block copolymers, heparin sulfate, dextran sulfate, hyaluronic acid, alginate, tripolyphosphate (TPP), oligo(glutamic acid), a cytokine, a protein, a peptide, a growth factor, and a nucleic acid.

13. The method of claim 12, wherein the nucleic acid is selected from the group consisting of an antisense oligonucleotide, cDNA, genomic DNA, guide RNA, plasmid DNA, vector DNA, mRNA, miRNA, piRNA, shRNA, and siRNA.

14. The method of any of claims 3-13, wherein the first stream and/or the second stream further comprise one or more water-soluble therapeutic agents.

15. The method of claim 14, wherein the one or more water-soluble therapeutic agents are selected from the group consisting of a small molecule, carbohydrate, sugar, protein, peptide, nucleic acid, antibody or antibody fragment thereof, hormone, hormone receptor, receptor ligand, cytokine, and growth factor.

16. The method of any of claims 1-15, wherein the one or more water-soluble polyanionic polymers is plasmid DNA and the one or more water-soluble polycationic polymers is linear polyethylenimine (PEI) or a derivative thereof.

17. The method of any of claims 1-16, comprising a plasmid DNA concentration between about 25 to about 800 μg/mL.

18. The method of claim 17, wherein the plasmid concentration is selected from the group consisting of about 25 μg/mL, about 50 μg/mL, about 100 μg/mL, about 200 μg/mL, about 400 μg/mL, and about 800 μg/mL.

19. A uniform polyelectrolyte complex (PEC) nanoparticle or plurality of PEC nanoparticles generated from the method of any of claims 1-18.

20. The PEC nanoparticle of claim 19, wherein the nanoparticle has an average of about 1 to about 50 copies of pDNA per nanoparticle.

21. The PEC nanoparticle of claim 20, wherein the PEC nanoparticle has an average of about 1.3 to about 21.8 copies of pDNA per nanoparticle; about 1.3 to about 1.4 copies of pDNA per nanoparticle; about 1.3 to about 1.6 copies of pDNA per nanoparticle; about 1.3 to about 1.7 copies of pDNA per nanoparticle; about 1.3 to about 2.3 copies of pDNA per nanoparticle; about 1.3 to about 2.6 copies of pDNA per nanoparticle; about 1.3 to about 3.5 copies of pDNA per nanoparticle; about 1.3 to about 4.4 copies of pDNA per nanoparticle; about 1.3 to about 4.7 copies of pDNA per nanoparticle; about 1.3 to about 5.0 copies of pDNA per nanoparticle; about 1.3 to about 6.1 copies of pDNA per nanoparticle; about 1.3 to about 8.0 copies of pDNA per nanoparticle; about 1.3 to about 8.5 copies of pDNA per nanoparticle; about 1.3 to about 9.1 copies of pDNA per nanoparticle; about 1.3 to about 9.5 copies of pDNA per nanoparticle; about 1.3 copies of pDNA per nanoparticle; about 3.5 copies of pDNA per nanoparticle; about 4.4 copies of pDNA per nanoparticle; about 5.0 copies of pDNA per nanoparticle; about 6.1 copies of pDNA per nanoparticle; about 8.0 copies of pDNA per nanoparticle; about 8.1 copies of pDNA per nanoparticle; about 8.5 copies of pDNA per nanoparticle; about 9.1 copies of pDNA per nanoparticle; about 9.5 copies of pDNA per nanoparticle; about 1.3 to about 10.0 copies of pDNA per nanoparticle; about 1.3 to about 13.5 copies of pDNA per nanoparticle; or about 21.8 pDNA copies per nanoparticle.

22. The PEC nanoparticle of claim 20, wherein the PEC nanoparticle has one pDNA per nanoparticle.

23. The PEC nanoparticle of any of claims 19-22, wherein the nanoparticle has an average size between about 30 nm to about 130 nm.

24. The PEC nanoparticle of any of claims 19-23, wherein the one or more water-soluble polycationic polymers comprises polyethylenimine and the one or more water-soluble polyanionic polymers comprises plasmid DNA.

25. The PEC nanoparticle of any of claims 19-24, wherein the PEC nanoparticle has a ratio of amine in the polyethylenimine to phosphate in the plasmid DNA (N/P) between about 3 to about 10.

26. The PEC nanoparticle of claim 25, wherein the PEC nanoparticle has an N/P selected from the group consisting of about 3, about 4, about 5, about 6, about 7, about 8, about 9, and about 10.

27. The PEC nanoparticle of any of claims 19-26, wherein the PEC nanoparticle has a percentage of bound WEI to total WEI between about 50% to about 75%.

28. The PEC nanoparticle of any of claims 19-28, wherein the plurality of PEC nanoparticles has a polydispersity index (PDI) between about 0.1 and about 0.25.

29. The PEC nanoparticle of any of claims 19-28, wherein the nanoparticle has an apparent hydrodynamic density between about 60 Da/nm³ to about 80 Da/nm³.

30. A pharmaceutical formulation comprising the PEC nanoparticle or plurality of PEC nanoparticles of any of claims 19-29 in a pharmaceutically acceptable carrier.

31. The pharmaceutical formulation of claim 30, wherein the formulation comprises a lyophilized formulation.

32. The pharmaceutical formulation of claim 31, wherein the PEC nanoparticle or plurality of PEC nanoparticles exhibits long term stability at −20° C. for at least 9 months.
